(19) United States
(12) Patent Application Publication
(10) Pub. No.: US 2022/0340974 A1
(43) Pub. Date: Oct. 27, 2022

(54) Method of Predicting Requirement for Biologic Therapy

(71) Applicant: Queen Mary University of London, London (GB)

(72) Inventors: Costantino Pitzalis, Kent (GB); Myles J. Lewis, London (GB); Frances Clare Humby, London (GB)

(21) Appl. No.: 17/764,717

(22) PCT Filed: Sep. 30, 2020
(86) PCT No.: PCT/GB2020/052367

§ 371 Date: Mar. 29, 2022

(30) Foreign Application Priority Data
Sep. 30, 2019 (GB) 1914079.7

(51) Int. Cl. C12Q 1/6883

(52) U.S. Cl. CPC C12Q 1/6883; C12Q 2600/106; C12Q 2600/158

(57) ABSTRACT

A method for identifying a subject requiring treatment with a biologic therapy for rheumatoid arthritis, the method comprising the steps: (a) determining the level of one or more biomarkers in one or more samples obtained from the subject, wherein the one or more biomarkers are selected from Table 1; and (b) comparing the level of the one or more biomarkers to one or more corresponding reference values; wherein the levels of the one or more biomarkers compared to the corresponding reference values are indicative of the requirement for treatment with a biologic therapy for rheumatoid arthritis.

### FIELD OF THE INVENTION

The present invention relates to methods for predicting whether a subject will require biologic therapy for rheumatoid arthritis. The invention also relates to methods for treating a subject for rheumatoid arthritis.

### BACKGROUND TO THE INVENTION

Inflammatory arthritis is a prominent clinical manifestation in diverse autoimmune disorders including rheumatoid arthritis (RA), psoriatic arthritis (PsA), systemic lupus erythematosus (SLE), Sjogren's syndrome and polymyositis.

RA is a chronic inflammatory disease that affects approximately 0.5 to 1% of the adult population in northern Europe and North America. It is a systemic inflammatory disease characterised by chronic inflammation in the synovial membrane of affected joints, which ultimately leads to loss of daily function due to chronic pain and fatigue. The majority of patients also experience progressive deterioration of cartilage and bone in the affected joints, which may eventually lead to permanent disability. The long-term prognosis of RA is poor, with approximately 50% of patients experiencing significant functional disability within 10 years from the time of diagnosis. Life expectancy is reduced by an average of 3-10 years.

Inflammatory bone diseases, such as RA, are accompanied by bone loss around affected joints due to increased osteoclastic resorption. This process is mediated largely by increased local production of pro-inflammatory cytokines, of which tumour necrosis factor-alpha (TNF-α) is a major effector.

In RA specifically, an immune response is thought to be initiated/perpetuated by one or several antigens presenting in the synovial compartment, producing an influx of acute inflammatory cells and lymphocytes into the joint. Successive waves of inflammation lead to the formation of an invasive and erosive tissue called pannus. This contains proliferating fibroblast-like synoviocytes and macrophages that produce proinflammatory cytokines such as TNF-α and interleukin-1 (IL-1). Local release of proteolytic enzymes, various inflammatory mediators, and osteoclast activation contributes to much of the tissue damage. There is loss of articular cartilage and the formation of bony erosions. Surrounding tendons and bursa may become affected by the inflammatory process. Ultimately, the integrity of the joint structure is compromised, producing disability.

B cells are thought to contribute to the immunopathogenesis of RA, predominantly by serving as the precursors of autoantibody-producing cells but also as antigen presenting cells (APC) and pro-inflammatory cytokine producing cells. A number of autoantibody specificities have been identified including antibodies to Type II collagen and proteoglycans, as well as rheumatoid factors and most importantly anti citrullinated protein antibodies (ACPA). The generation of large quantities of antibody leads to immune complex formation and the activation of the complement cascade. This in turn amplifies the immune response and may culminate in local cell lysis.

Current standard therapies for RA which are used to modify the disease process and to delay joint destruction are known as disease modifying anti-rheumatic drugs (DMARDs). Methotrexate, leflunomide and sulfasalazine are traditional DMARDs and are often effective as first-line treatment.

Biologic agents designed to target specific components of the immune system that play roles in RA are also used as therapeutics. There are various groups of biologic treatments for RA, including TNF-α inhibitors (etanercept, infliximab and adalimumab), human IL-1 receptor antagonists (anakinra), and selective co-stimulation modulators (abatacept).

The introduction of ACR/EULAR RA classification criteria have impacted positively on early diagnosis and treatment of RA leading to better outcomes. By the same token, broader criteria have led to the inclusion of patients with milder and more heterogeneous disease. This, together with the inability to precisely predict disease prognosis and treatment response at the individual patent level, emphasises the need to identify patients at risk of accelerated structural damage progression and fast-track aggressive/biologic therapies to patients with poor prognosis.

The identification at disease onset of patients who are unlikely to respond to csDMARDs, remains a major unmet need. The capacity to refine early clinical classification criteria and the ability to identify patients who subsequently require biologic therapy at disease onset would offer the opportunity to stratify therapeutic intervention to the patients most in need.

Accordingly, there is a need for methods of predicting whether a subject will require biologic therapy for rheumatoid arthritis, in particular at disease onset. There is also a need for methods for treating a subject for rheumatoid arthritis.

### SUMMARY OF THE INVENTION

The present invention addresses the above prior art problems by providing methods for identifying a subject requiring treatment with a biologic therapy for rheumatoid arthritis, together with methods for treating a subject so identified, as described in the claims.

The present inventors have studied the largest biopsy-driven early inflammatory arthritis cohort to date (200 patients) and, through a detailed synovial cellular and molecular characterisation, refined ACR/EULAR disease classification. In addition, the inventors have identified synovial pathobiological markers associated with the lympho-myeloid pathotype and the requirement of biologic therapy at 12 months. Notably, these findings are independent from the time of diagnosis within the first 12 months of symptoms initiation, suggesting that the so called “window of opportunity” is wider than 6 months and early stratification of biologic therapies according to poor prognostic synovial pathobiological subtypes at disease onset may improve the outcome of these patients. The integration of synovial pathobiological markers into a logistic regression model improves the prediction accuracy from 78.8% to 89-90% and enables the identification at disease onset of patients who subsequently require biologic therapy. The inventors' approach enables biologic therapies to be started early in patients with poor prognosis.

In one aspect, the invention provides a method for identifying a subject requiring treatment with a biologic therapy for rheumatoid arthritis, the method comprising the steps: (a) determining the level of one or more biomarkers in one or more samples obtained from the subject, wherein the one or more biomarkers are selected from Table 1; and (b) comparing the level of the one or more biomarkers to one or more corresponding reference values; wherein the levels of the one or more biomarkers compared to the corresponding reference values are indicative of the requirement for treatment with a biologic therapy for rheumatoid arthritis.

In another aspect, the invention provides a method for identifying a subject requiring treatment with a therapy for rheumatoid arthritis other than, or in addition to, a Disease-Modifying Anti-Rheumatic Drug (DMARD), the method comprising the steps: (a) determining the level of one or more biomarkers in one or more samples obtained from the subject, wherein the one or more biomarkers are selected from Table 1; and (b) comparing the level of the one or more biomarkers to one or more corresponding reference values; wherein the levels of the one or more biomarkers compared to the corresponding reference values are indicative of the requirement for treatment with a therapy for rheumatoid arthritis other than, or in addition to, a DMARD.

In another aspect, the invention provides a method for identifying a subject that is likely to be DMARD-refractory, the method comprising the steps: (a) determining the level of one or more biomarkers in one or more samples obtained from the subject, wherein the one or more biomarkers are selected from Table 1; and (b) comparing the level of the one or more biomarkers to one or more corresponding reference values; wherein the levels of the one or more biomarkers compared to the corresponding reference values are indicative of the subject being DMARD-refractory.

In one aspect, the invention provides a method for selecting a therapy for a subject having or suspected of having rheumatoid arthritis, the method comprising the steps: (a) determining the level of one or more biomarkers in one or more samples obtained from the subject, wherein the one or more biomarkers are selected from Table 1; and (b) comparing the level of the one or more biomarkers to one or more corresponding reference values; wherein the levels of the one or more biomarkers compared to the corresponding reference values are indicative of the requirement for treatment with a biologic therapy for rheumatoid arthritis.

In another aspect, the invention provides a method for identifying a subject for which treatment of rheumatoid arthritis solely with a Disease-Modifying Anti-Rheumatic Drug (DMARD) is likely to be ineffective, the method comprising the steps: (a) determining the level of one or more biomarkers in one or more samples obtained from the subject, wherein the one or more biomarkers are selected from Table 1; and (b) comparing the level of the one or more biomarkers to one or more corresponding reference values; wherein the levels of the one or more biomarkers compared to the corresponding reference values are indicative of treatment of rheumatoid arthritis solely with a DMARD being ineffective.

In some embodiments, the one or more biomarkers comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or all 72 biomarkers from Table 1.

In some embodiments, the one or more biomarkers comprise all biomarkers from Table 1.

In some embodiments, the one or more biomarkers consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or all 72 biomarkers from Table 1.

In some embodiments, the one or more biomarkers consist of all biomarkers from Table 1.

In some embodiments, the one or more biomarkers are selected from Table 2 and the levels of the one or more biomarkers are increased compared to the corresponding reference values.

In some embodiments, the one or more biomarkers comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or all 49 biomarkers from Table 2.

In some embodiments, the one or more biomarkers comprise all biomarkers from Table 2.

In some embodiments, the one or more biomarkers consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or all 49 biomarkers from Table 2.

In some embodiments, the one or more biomarkers consist of all biomarkers from Table 2.

In some embodiments, the one or more biomarkers are selected from Table 3 and the levels of the one or more biomarkers are decreased compared to the corresponding reference values.

In some embodiments, the one or more biomarkers comprise at least 2, 3, 4, 5, 6, 7, 8, 9, 10, 11, 12, 13, 14, 15, 16, 17, 18, 19, 20, 21, 22 or all 23 biomarkers from Table 3.

In some embodiments, the one or more biomarkers comprise all biomarkers from Table 3.

In some embodiments, the one or more biomarkers consist of 2, 3, 4, 5, 6, 7, 8, 9, 10, 11, 12, 13, 14, 15, 16, 17, 18, 19, 20, 21, 22 or all 23 biomarkers from Table 3.

In some embodiments, the one or more biomarkers consist of all biomarkers from Table 3.

In some embodiments, the one or more biomarkers comprise one or more of GPR114, CSF1, MMP3, IL20 and MMP10. In some embodiments, the one or more biomarkers comprise GPR114, CSF1, MMP3, IL20 and MMP10.

In some embodiments, the one or more biomarkers comprise one or more of GPR114, CSF1, MMP3, IL20, MMP10 and NOG. In some embodiments, the one or more biomarkers comprise GPR114, CSF1, MMP3, IL20, MMP10 and NOG.

In some embodiments, the one or more biomarkers comprise one or more of GPR114, IL8, CSF1, MMP3, LTB, HIVEP1, IL20, UBASH3A and MMP10. In some embodiments, the one or more biomarkers comprise GPR114, IL8, CSF1, MMP3, LTB, HIVEP1, IL20, UBASH3A and MMP10.

In some embodiments, the one or more biomarkers comprise one or more of GPR114, IL8, CSF1, MMP3, HIVEP1, IL20, MMP10, NOG and IFNB1. In some embodiments, the one or more biomarkers comprise GPR114, IL8, CSF1, MMP3, HIVEP1, IL20, MMP10, NOG and IFNB1.

In some embodiments, the method further comprises determining one or more clinical covariates of the subject and comparing the one or more clinical covariates to one or more reference values. The clinical covariates may, for example be selected from the group consisting of Disease Activity Score (DAS), DAS28, baseline pathotype, C-reactive protein and tender joint count (TJC).

In some embodiments, the method further comprises determining the C-reactive protein and DAS28 clinical covariates of the subject and comparing each clinical covariate to one or more reference values. In some embodiments, the method further comprises determining the pathotype, C-reactive protein, TJC and DAS28 clinical covariates of the subject and comparing each clinical covariate to one or more reference values.

In some embodiments, the one or more biomarkers comprise one or more of GPR114, CSF1, MMP3, IL20 and MMP10, and the method further comprises determining the C-reactive protein and DAS28 clinical covariates of the subject and comparing each clinical covariate to one or more reference values. In some embodiments, the one or more biomarkers comprise GPR114, CSF1, MMP3, IL20 and MMP10, the method further comprises determining the C-reactive protein and DAS28 clinical covariates of the subject and comparing each clinical covariate to one or more reference values.

In some embodiments, the one or more biomarkers comprise one or more of GPR114, CSF1, MMP3, IL20, MMP10 and NOG, and the method further comprises determining the pathotype, C-reactive protein, TJC and DAS28 clinical covariates of the subject and comparing each clinical covariate to one or more reference values. In some embodiments, the one or more biomarkers comprise GPR114, CSF1, MMP3, IL20, MMP10 and NOG, and the method further comprises determining the pathotype, C-reactive protein, TJC and DAS28 clinical covariates of the subject and comparing each clinical covariate to one or more reference values.

In some embodiments, the one or more biomarkers comprise one or more of GPR114, IL8, CSF1, MMP3, LTB, HIVEP1, IL20, UBASH3A and MMP10, and the method further comprises determining the C-reactive protein and DAS28 clinical covariates of the subject and comparing each clinical covariate to one or more reference values. In some embodiments, the one or more biomarkers comprise GPR114, IL8, CSF1, MMP3, LTB, HIVEP1, IL20, UBASH3A and MMP10, and the method further comprises determining the C-reactive protein and DAS28 clinical covariates of the subject and comparing each clinical covariate to one or more reference values.

In some embodiments, the one or more biomarkers comprise one or more of GPR114, IL8, CSF1, MMP3, HIVEP1, IL20, MMP10, NOG and IFNB1, and the method further comprises determining the pathotype, C-reactive protein, TJC and DAS28 clinical covariates of the subject and comparing each clinical covariate to one or more reference values. In some embodiments, the one or more biomarkers comprise GPR114, IL8, CSF1, MMP3, HIVEP1, IL20, MMP10, NOG and IFNB1, and the method further comprises determining the pathotype, C-reactive protein, TJC and DAS28 clinical covariates of the subject and comparing each clinical covariate to one or more reference values.

In some embodiments, the one or more biomarkers comprise one or more of GPR114, IL8, CSF1, MMP3, LTB, HIVEP1, IL20, UBASH3A, MMP10, NOG and IFNB1. In some embodiments, the one or more biomarkers comprise GPR114, IL8, CSF1, MMP3, LTB, HIVEP1, IL20, UBASH3A, MMP10, NOG and IFNB1. In some embodiments, the one or more biomarkers comprise GPR114, IL8, CSF1, MMP3, LTB, HIVEP1, IL20, UBASH3A, MMP10, NOG and IFNB1, and the method further comprises determining the pathotype, C-reactive protein and TJC (and optionally DAS28) clinical covariates of the subject and comparing each clinical covariate to one or more reference values.

Exemplary biomarkers and/or clinical covariates for use in the methods described herein are those described in Example 1 and/or FIG. 6B.

In some embodiments, the step of determining the levels of the one or more biomarkers comprises determining the levels of gene expression of the one or more biomarkers.

In some embodiments, the level is a nucleic acid level. In some embodiments, the nucleic acid level is an mRNA level.

In some embodiments, the level of the one or more biomarkers is determined by direct digital counting of nucleic acids, RNA-seq, RT-qPCR, qPCR, multiplex qPCR or RT-qPCR, microarray analysis, or a combination thereof.

In some embodiments, the level is a protein level.

In some embodiments, the level of the one or more biomarkers is determined by an immunoassay, liquid chromatography-mass spectrometry (LC-MS), nephelometry, aptamer technology, or a combination thereof.

In preferred embodiments, the subject has not been previously treated for rheumatoid arthritis. In preferred embodiments, the subject is treatment naïve for Disease-Modifying Anti-Rheumatic Drugs (DMARDs) and/or steroids.

In some embodiments, the subject has not been previously treated with a Disease-Modifying Anti-Rheumatic Drug (DMARD). In some embodiments, the subject has not been previously treated with a biologic therapy for rheumatoid arthritis. In preferred embodiments, the subject has not been previously treated with a Disease-Modifying Anti-Rheumatic Drug (DMARD) or a biologic therapy for rheumatoid arthritis.

In some embodiments, the subject is suspected of having rheumatoid arthritis.

In some embodiments, the subject has presented one or more symptoms of rheumatoid arthritis for less than 1 year (e.g. less than 9, 8, 7, 6, 5, 4, 3, 2 or 1 months).

In some embodiments, the sample is a synovial sample. In some embodiments, the sample is a synovial tissue sample or a synovial fluid sample.

In some embodiments, the sample is obtained by synovial biopsy, preferably ultrasound-guided synovial biopsy.

In some embodiments, the method further comprises administering to the subject a biologic therapy for rheumatoid arthritis when the subject is identified as requiring treatment with a biologic therapy for rheumatoid arthritis; requiring treatment with a therapy for rheumatoid arthritis other than, or in addition to, a Disease-Modifying Anti-Rheumatic Drug (DMARD); or being DMARD-refractory.

In some embodiments, the method further comprises administering to the subject a therapeutic agent other than, or in addition to, a Disease-Modifying Anti-Rheumatic Drug (DMARD) when the subject is identified as requiring treatment with a biologic therapy for rheumatoid arthritis; requiring treatment with a therapy for rheumatoid arthritis other than, or in addition to, a Disease-Modifying Anti-Rheumatic Drug (DMARD); or being DMARD-refractory.

In some embodiments, the biologic therapy is a B cell antagonist, a Janus kinase (JAK) antagonist, a tumour necrosis factor (TNF) antagonist, a decoy TNF receptor, a T cell costimulatory signal antagonist, an IL-1 receptor antagonist, an IL-6 receptor antagonist, or a combination thereof.

In some embodiments, the biologic therapy is an anti-TNF-alpha therapy or an anti-CD20 therapy.

In some embodiments, the anti-TNF-alpha therapy comprises an anti-TNF-alpha antibody, preferably adalimumab.

In some embodiments, the anti-CD20 therapy comprises an anti-CD20 antibody, preferably rituximab.

In some embodiments, the biologic therapy is selected from the group consisting of adalimumab, infliximab, certolizumab pegol, golimumab, rituximab, ocrelizumab, veltuzumab, ofatumumab, tocilizumab and tofacitinib, or a combination thereof.

In some embodiments, the DMARD is selected from the group consisting of methotrexate, hydroxychloroquine, sulfasalazine, leflunomide, azathioprine, cyclophosphamide, cyclosporine and mycophenolate mofetil, or a combination thereof.

In some embodiments, the method further comprises the step of determining whether the subject exhibits a lympho-myeloid pathotype.

In another aspect, the invention provides a method of treating rheumatoid arthritis, the method comprising administering to the subject an effective amount of a biologic therapy for rheumatoid arthritis, wherein the subject has been identified as having a requirement for treatment with a biologic therapy for rheumatoid arthritis; having a requirement for treatment with a therapy for rheumatoid arthritis other than, or in addition to, a Disease-Modifying Anti-Rheumatic Drug (DMARD); or being DMARD-refractory, by a method of any preceding claim.

### DESCRIPTION OF THE DRAWINGS

FIG. 1

Baseline Patient Demographics. (A) Baseline classification of patients. 200 patients were classified into RA1987 vs undifferentiated arthritis (UA). RA 2010 ACR/EULAR Criteria was then applied to UA patients. Final 3 groups obtained showed 47 patients UA (RA 1987−/RA2010−), RA 2010 (RA1987−/RA2010+), RA 1987 (RA1987+/RA2010+). (B) Demographics according to classification criteria. Data are presented as mean (SD, standard deviation) for continue variables and frequency and percentages for categorical variables. Baseline characteristics between the 3 groups were compared using Kruskal-Wallis or Fisher's exact test as appropriate. For post hoc comparison, Dunn tests were run and p-value from pairwise comparison reported in the last 3 columns of the table. ESR: Erythrocyte sedimentation rate; CRP: C-reactive protein; 28TJC: 28 tender joint count; 28SJC: 28 swollen joint count; DAS28: Disease Activity Score 28 joints; RF titre: Rheumatoid factor titre (IU/ml); ACPA Titre: Anti-citrullinated protein antibody titre (IU/L); RF +ve: rheumatoid factor serum positive (>15 IU/L); ACPA +ve: Anti-citrullinated protein antibody (>20 IU/L).

FIG. 2

Patient demographics and disease activity: comparison between pathotypes. (A) Number of biopsy procedures per joint MCP (Metacarpophalangeal), MTP (Metatarsophalangeal), PIP (Proximal Inter phalangeal). (B) Representative images of synovial pathotypes. H&E: Haematoxylin & Eosin. Sections underwent immunohistochemical staining and semi-quantitative scoring (0-4) to determine the degree of CD20+ B cells, CD3+ T cells, CD68+ lining (I) and sublining (sl) macrophage and CD138+ plasma cell infiltration. Sections were categorised into three pathotypes: (i) Pauci-iumne (CD68 SL<2 and or CD3, CD20, CD138<1), (ii) Diffuse-Myeloid: (CD68SL>2, CD20<1 and or CD3>1) and (iii) Lympho-Myeloid: (grade 2-3 CD20+ aggregates, CD20>2). Arrow heads indicate positive stain cells. Empty arrows indicate B cell aggregates. (C) Demographic Analysis by Pathotype. Data are presented as mean and standard deviation (SD) for numerical variables and frequency and percentage for categorical variables. Baseline characteristics between the 3 pathotypes were compared using a Kruskall-Wallis test and Fisher-test (RF and ACPA positivity) as appropriate. Post hoc analysis for significant differences using Dunn test for multiple comparison. A P-value of <0.05 was considered statistically significant. (D) Pathotype according to disease duration (months) at diagnosis. Absolute values (N) and percentage. A P-value of <0.05 was considered statistically significant.

FIG. 3

Variation in synovial pathobiology according to clinical classification of patients. (A) Baseline clinical classification compared with pathotype. Baseline subgroups (RA 1987, RA2010 and UA) were compared with pathotype. Fisher test used for analysis. (B) Immune cell infiltration for each clinical subgroup. Kruskal-Wallis test for comparison between 3 groups. Post hoc analysis for significant differences using Dunn test for multiple comparison. (C-E) Gene expression analysis for comparison between subgroups. T-test for comparison and Volcano plot for representative image. Positive values represent upregulation and negative values downregulation. Green circles above green horizontal line represents non-corrected for multiple analysis expressed genes between groups. Red circles above red line represents corrected p-values (Benjamini-Hochberg method) for multiple analysis. (C) Volcano plot RA 1987 vs RA 2010: Difference in gene expression between patient fulfilling RA 1987 ACR criteria and RA 2010 ACR/EULAR Criteria. (D) Volcano plot RA 1987 vs UA: Difference in gene expression between patient fulfilling RA 1987 ACR criteria and Undifferentiated Arthritis. (E) Volcano plot RA 2010 vs UA: differences in gene expression between patient fulfilling RA 2010 ACR/EULAR criteria and UA.

FIG. 4

Disease evolution. (A) Patient classification after 12 months follow up. Disease outcome after 12 months of follow up for each of the initial baseline subgroups (RA1987/RA2010/UA). Disease evolution classified as self-limiting or persistent disease. Other diagnosis as described for those who were re-classified after 1 year form UA cohort. (B) Disease evolution by subgroups. Disease evolution was compared with Baseline subgroups (RA 1987, RA2010 and UA). Fisher test used for analysis. (C) Disease evolution by pathotype. Disease evolution was compared with pathotype (Pauci-imune vs Diffuse-Myeloid vs Lympho-Myeloid. Fisher test used for analysis. A P-value of <0.05 was considered statistically significant.

FIG. 5

(A) Comparison between diagnostic subgroups and treatment outcome at 12 month follow up. Treatment required was divided in 3 groups: (i) No treatment; (ii) csDMARDs only, (iii) csDMARDs+/−Biologics. Fisher test for analysis. (B) Comparison between pathotype and treatment outcome at 12 months. (C) Gene expression analysis, represented in a Volcano plot comparison between patient requiring Biologics vs non-biologic group. T-test comparison for gene difference expression between groups. Positive values represents upregulation and negative values downregulation. An adjusted (Benjamini-Hochberg correction for multiple analysis) P-value of <0.01 was considered statistically significant, represented as dots above red line. Green dots above green line for gene expression significance when no correction applied for multiple analysis (P value <0.05). (D) Treatment outcome according to baseline disease duration. Fisher test for analysis. (E) Pathotype according to baseline disease duration for Biologic patient cohort. Fisher test for analysis. A P-value of <0.05 was considered statistically significant unless otherwise stated.

FIG. 6

Prediction model. (A-B) Identification of clinical and gene expression features predictive of biologic therapy use at 1 year. Logistic regression, coupled with backward and stepwise model selection was applied to baseline clinical parameters against a dependent variable of Biologic therapy use or not at 12 months to select which clinical covariate contributed the most to the prediction. Selected covariates (119 genes+4 clinical covariates) were entered simultaneously into a logistic model with an L1 regularization penalty (LASSO) in order to determine the optimal sparse prediction model. A similar predictive performance of the model when clinical was seen when results were penalized (blue dashed line, FIG. 6A) than when they were not penalized (red dotted line, FIG. 6A) with a slightly different set of selected covariates (FIG. 6B). FIG. 6B shows the non-zero weights associated with the final variables selected by the LASSO regression. The grey spaces represent the variables that were not selected by the model. (C-D) Lambda training curve from the final glmnet fitted model. The red dots represent mean binomial deviance using 10-fold cross-validation. The error bars represent standard error of binomial deviance. The vertical dotted lines indicate minimum binomial deviance (λmin) and a more regularised model for which the binomial deviance error is within one standard error of the minimum binomial deviance (λ1se). λmin was selected, corresponding to 11 non-zero coefficients in the final model for the LASSO where clinical were penalised (FIG. 6C) and 13 non-zero coefficients in the final model for the LASSO where clinical were not penalised (FIG. 6D).

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Rheumatoid Arthritis (RA)

Rheumatoid arthritis (RA) is a chronic, systemic inflammatory disorder that may affect many tissues and organs, but principally attacks synovial joints. It is a disabling and painful condition, which can lead to substantial loss of functioning and mobility if not adequately treated.

The disease process involves an inflammatory response of the synovium, secondary to massive immune cell infiltration and proliferation of synovial cells, excess synovial fluid, and the development of fibrous tissue (pannus) in the synovium that attacks the cartilage and sub-chondral bone. This often leads to the destruction of articular cartilage and the formation of bone erosions with secondary ankylosis (fusion) of the joints. RA can also produce diffuse inflammation in the lungs, the pericardium, the pleura, the sclera, and also nodular lesions, most commonly in subcutaneous tissue. RA is considered a systemic autoimmune disease as autoimmunity plays a pivotal role in its chronicity and progression.

A number of cell types are involved in the aetiology of RA, including T cells, B cells, monocytes, macrophages, dendritic cells and synovial fibroblasts. Autoantibodies known to be associated with RA include those targeting Rheumatoid factor (RF) and anti-citrullinated protein antibodies (ACPA).

RA Therapy

A typical patient with newly diagnosed RA is often treated initially with nonsteroidal anti-inflammatory drugs and disease-modifying anti-rheumatic drugs (DMARDs), such as hydroychloroquine, sulfasalazine, leflunomide or methotrexate (MTX), alone or in combinations. Patients who do not respond to general DMARDs may be termed DMARD-refractory.

DMARD-refractory patients are traditionally often progressed to biological therapeutic agents, for example TNF-α antagonists such as Adalimumab, Etanercept, Golimumab and Infliximab. Patients who do not respond to TNF-α antagonist therapy may be termed TNF-α antagonist-refractory or inadequate responders (ir).

The capacity, provided by the present invention, to refine early clinical classification criteria and the ability to identify patients who will require biologic therapy at disease onset offers the opportunity to stratify therapeutic intervention to the patients most in need and enables biologic therapies to be started early in patients with poor prognosis.

The term “biologic therapy”, as used herein, may refer to protein agents that enable treatment for rheumatoid arthritis. Example biologic therapies for rheumatoid arthritis are well known in the art, and suitable biologic therapies may be readily selected by the skilled person.

In some embodiments, the biologic therapy for rheumatoid arthritis is an antibody.

In some embodiments, the biologic therapy is a B cell antagonist, a Janus kinase (JAK) antagonist, a tumour necrosis factor (TNF) antagonist, a decoy TNF receptor, a T cell costimulatory signal antagonist, an IL-1 receptor antagonist, an IL-6 receptor antagonist, or a combination thereof.

In some embodiments, the biologic therapy is an anti-TNF-alpha therapy or an anti-CD20 therapy.

In some embodiments, the anti-TNF-alpha therapy comprises an anti-TNF-alpha antibody, preferably adalimumab.

In some embodiments, the anti-CD20 therapy comprises an anti-CD20 antibody, preferably rituximab.

In some embodiments, the biologic therapy is selected from the group consisting of adalimumab, infliximab, certolizumab pegol, golimumab, rituximab, ocrelizumab, veltuzumab, ofatumumab, tocilizumab and tofacitinib, or a combination thereof.

Anti-TNF-Alpha Therapy The term “anti-TNF-alpha therapy”, as used herein, is intended to encompass the use of therapeutic substances whose mechanism of action involves suppressing the physiological response to TNF-alpha.

In particular, anti-TNF-alpha therapies include TNF-inhibitors, which may act by binding to TNF-alpha and inhibiting its ability to bind to its receptors. Examples of TNF-inhibitors include anti-TNF-alpha antibodies, and the fusion protein etanercept.

Examples of anti-TNF-alpha antibodies include adalimumab (Humira), infliximab (Remicade), certolizumab pegol (Cimzia) and golimumab (Simponi).

Adalimumab is a monoclonal antibody sold under the trade name Humira and used to treat conditions including rheumatoid arthritis, psoriatic arthritis, ankylosing spondylitis, Crohn's disease, ulcerative colitis, chronic psoriasis, hidradenitis suppurativa, and juvenile idiopathic arthritis.

Certolizumab is a fragment of a monoclonal antibody sold as certolizumab pegol under the trade name Cimzia. It is used for the treatment of Crohn's disease, rheumatoid arthritis, psoriatic arthritis and ankylosing spondylitis.

Anti-CD20 Therapy

The term “anti-CD20 therapy”, as used herein, is intended to encompass the use of therapeutic substances whose mechanism of action involves binding to CD20. The anti-CD20 therapy may interfere with or inhibit the development and/or function of B cells. The anti-CD20 therapy may cause B cell depletion or the inhibition of B cell development and maturation.

In some embodiments, the anti-CD20 therapy comprises an anti-CD20 antibody (e.g. an anti-CD20 monoclonal antibody), for example Rituximab.

Antibodies directed against CD20 may bind to the target antigen and kill a cell on the surface of which it is expressed by initiating a mixture of apoptosis, complement dependent cytotoxicity (CDC) and antibody-dependent cell-mediated cellular cytotoxicity (ADCC).

In some embodiments, the anti-CD20 therapy is selected from the group consisting of Rituximab, Ocrelizumab, Veltuzumab and Ofatumumab.

In preferred embodiments, the anti-CD20 therapy is Rituximab.

Rituximab is a chimeric mouse/human immunoglobulin G1 (IgG1) monoclonal antibody to CD20 that stimulates B cell destruction upon binding to CD20. Rituximab depletes CD20 surface-positive naïve and memory B cells from the blood, bone marrow and lymph nodes via mechanisms which include antibody-dependent cellular cytotoxicity (ADCC), complement dependent cytotoxicity (CDC). It does not affect CD20-negative early B cell lineage precursor cells and late B lineage plasma cells in the bone marrow.

Ocrelizumab is a humanised anti-CD20 monoclonal antibody that causes CD20+ B cell depletion following binding to CD20 via mechanisms including ADCC and CDC.

Veltuzumab is a humanised, second-generation anti-CD20 monoclonal antibody that causes CD20+ B cell depletion following binding to CD20 via mechanisms including ADCC and CDC.

Ofatumumab is a human monoclonal IgG1 antibody to CD20 and may inhibit early-stage B lymphocyte activation. Ofatumumab targets a different epitope located closer to the N-terminus of CD20 compared to the epitope targeted by rituximab and includes an extracellular loop, as it binds to both the small and large loops of the CD20 molecule. Ofatumumab stimulates B cell destruction through ADCC and CDC pathways.

B Cells

B cells play a central role in the pathogenesis of RA.

Immature B cells are produced in the bone marrow. After reaching the IgM⁺ immature stage in the bone marrow, these immature B cells migrate to secondary lymphoid tissues (such as the spleen, lymph nodes) where they are called transitional B cells, and some of these cells differentiate into mature B lymphocytes and possibly plasma cells.

B cells may be defined by a range of cell surface markers which are expressed at different stages of B cell development and maturation (see table below). These B cell markers may include CD19, CD20, CD22, CD23, CD24, CD27, CD38, CD40, CD72, CD79a and CD79b, CD138 and immunoglobulin (Ig).

| Compartment | Cell type | Markers |
|---|---|---|
| Bone marrow | Stem cell | |
| | Pro-B cell | CD19⁺ CD20⁻ Ig⁻ |
| | Pre-B cell | CD19⁺ CD20⁺ Ig⁻ |
| | Immature B cell | CD19⁺ CD20⁺ Ig⁺ |
| Peripheral | Naïve B cell | CD19⁺ CD20⁺ Ig⁺ CD38^{+/-} |
| compartments | Naïve activated | CD19⁺ CD20⁺ Ig⁺ CD38⁺ |
| | B cell | |
| | GC B cell | CD19⁺ CD20⁺ Ig⁺ CD38⁺⁺ |
| | Post-GC B cell | CD19⁺ CD20⁺ Ig⁺ CD38⁺ |
| | Memory B cell | CD19⁺ CD20⁺ Ig^{+/-} CD27⁺ |
| | | IgM/IgG/IgA⁺ CD38⁻ |
| | Plasma blast | CD19⁺ CD20⁻ Ig^{+/-} CD27⁺⁺ CD38⁺⁺ |
| Bone marrow | Plasma cell | CD19^{+/-} CD20⁻ Ig⁻ CD27⁺⁺ CD38⁺⁺⁺ |
| | | CD138⁺ |

Immunoglobulins (Ig) are glycoproteins belonging to the immunoglobulin superfamily which recognise foreign antigens and facilitate the humoral response of the immune system. Ig may occur in two physical forms, a soluble form that is secreted from the cell, and a membrane-bound form that is attached to the surface of a B cell and is referred to as the B cell receptor (BCR). Mammalian Ig may be grouped into five classes (isotypes) based on which heavy chain they possess. Immature B cells, which have never been exposed to an antigen, are known as naïve B cells and express only the IgM isotype in a cell surface bound form. B cells begin to express both IgM and IgD when they reach maturity—the co-expression of both these immunoglobulin isotypes renders the B cell “mature” and ready to respond to antigen. B cell activation follows engagement of the cell bound antibody molecule with an antigen, causing the cell to divide and differentiate into an antibody producing plasma cell. In this activated form, the B cell starts to produce antibody in a secreted form rather than a membrane-bound form. Some daughter cells of the activated B cells undergo isotype switching to change from IgM or IgD to the other antibody isotypes, IgE, IgA or IgG, that have defined roles in the immune system.

CD19 is expressed by essentially all B-lineage cells and regulates intracellular signal transduction by amplifying Src-family kinase activity.

CD20 is a mature B cell-specific molecule that functions as a membrane embedded Ca²⁺ channel. Expression of CD20 is restricted to the B cell lineage from the pre-B-cell stage until terminal differentiation into plasma cells.

CD22 functions as a mammalian lectin for α2,6-linked sialic acid that regulates follicular B-cell survival and negatively regulates signalling.

CD23 is a low-affinity receptor for IgE expressed on activated B cells that influences IgE production.

CD24 is a GPI-anchored glycoprotein which was among the first pan-B-cell molecules to be identified.

CD27 is a member of the TNF-receptor superfamily. It binds to its ligand CD70, and plays a key role in regulating B-cell activation and immunoglobulin synthesis. This receptor transduces signals that lead to the activation of NF-κB and MAPK8/JNK.

CD38 is also known as cyclic ADP ribose hydrolase. It is a glycoprotein that also functions in cell adhesion, signal transduction and calcium signalling and is generally a marker of cell activation.

CD40 serves as a critical survival factor for germinal centre (GC) B cells and is the ligand for CD154 expressed by T cells.

CD72 functions as a negative regulator of signal transduction and as the B-cell ligand for Semaphorin 4D (CD100).

CD79a/CD79b dimer is closely associated with the B-cell antigen receptor, and enables the cell to respond to the presence of antigens on its surface. The CD79a/CD79b dimer is present on the surface of B-cells throughout their life cycle, and is absent on all other healthy cells.

CD138 is also known as Syndecan 1. Syndecans mediate cell binding, cell signalling and cytoskeletal organisation. CD138 may be useful as a cell surface marker for plasma cells.

Response to Therapies in RA Patients

Methods of assessing a subject's response to a therapy for rheumatoid arthritis are known in the art and would be familiar to a skilled person.

By way of example, well known measures of disease activity in RA include the Disease Activity Score (DAS), a modified version DAS28, and the DAS-based EULAR response criteria.

Biomarkers

The present invention provides a method for identifying a subject requiring treatment with a biologic therapy for rheumatoid arthritis, the method comprising the steps:
(a) determining the level of one or more biomarkers in one or more samples obtained from the subject, wherein the one or more biomarkers are selected from Table 1; and
(b) comparing the level of the one or more biomarkers to one or more corresponding reference values;

wherein the levels of the one or more biomarkers compared to the corresponding reference values are indicative of the requirement for treatment with a biologic therapy for rheumatoid arthritis.

In some embodiments, the one or more biomarkers comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or all 72 biomarkers from Table 1.

In some embodiments, the one or more biomarkers comprise all 72 biomarkers from Table 1.

In some embodiments, the one or more biomarkers consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or all 72 biomarkers from Table 1.

In some embodiments, the one or more biomarkers consist of all 72 biomarkers from Table 1.

TABLE 1
Biomarkers with significant differential regulation of gene expression
(Biologic vs No Biologic Treatment at 12m).

| | | | Exemplary |
|---|---|---|---|
| | | NCBI | nucleic acid |
| | | Gene | sequence (NCBI |
| Biomarker | Full Gene Name | ID | Accession No.) |
| GPR114 | Adhesion G protein-coupled | 221188 | NM_001304376.3 |
| | receptor G5 | | |
| CCL19 | C-C motif chemokine ligand | 6363 | NM_006274.3 |
| | 19 | | |
| MMP3 | Matrix metallopeptidase 3 | 4314 | NM_002422.5 |
| MMP1 | Matrix metallopeptidase 1 | 4312 | NM_001145938.2 |
| CXCL13 | C-X-C motif chemokine | 10563 | NM_001371558.1 |
| | ligand 13 | | |
| CD79A | CD79a molecule | 973 | NM_001783.4 |
| TNFRSF17 | TNF receptor superfamily | 608 | NM_001192.3 |
| | member 17 | | |
| IGJ | Joining chain of multimeric | 3512 | NM_144646.4 |
| | IgA and IgM | | |
| ZBP1 | Z-DNA binding protein 1 | 81030 | NM_001160417.2 |
| SLAMF7 | SLAM family member 7 | 57823 | NM_001282588.1 |
| PIM2 | Pim-2 proto-oncogene, | 11040 | NM_006875.4 |
| | serine/threonine kinase | | |
| CXCL1 | C-X-C motif chemokine | 2919 | NM_001511.4 |
| | ligand 1 | | |
| FCRL5 | Fc receptor like 5 | 83416 | NM_001195388.2 |
| MS4A1 | Membrane spanning 4- | 931 | NM_021950.3 |
| | domains A1 | | |
| PLA2G2D | Phospholipase A2 group IID | 26279 | NM_001271814.1 |
| KIAA0125 | Family with sequence | 9834 | NR_026800.2 |
| | similarity 30 member A | | |
| CD19 | CD19 molecule | 930 | NM_001178098.2 |
| MMP10 | Matrix metallopeptidase 10 | 4319 | NM_002425.3 |
| CD3E | CD3e molecule | 916 | NM_000733.4 |
| IL2RG | Interleukin 2 receptor subunit | 3561 | NM_000206.2 |
| | gamma | | |
| SEL1L3 | SEL1L family member 3 | 23231 | NM_001297592.2 |
| KCNA3 | Potassium voltage-gated | 3738 | NM_002232.5 |
| | channel subfamily A member | | |
| | 3 | | |
| TNFRSF13C | TNF receptor superfamily | 115650 | NM_052945.3 |
| | member 13C | | |
| SIRPG | Signal regulatory protein | 55423 | NM_001039508.1 |
| | gamma | | |
| TMEM156 | Transmembrane protein 156 | 80008 | NM_001303228.2 |
| CD40LG | CD40 ligand | 959 | NM_000074.3 |
| DEF6 | DEF6 guanine nucleotide | 50619 | NM_022047.4 |
| | exchange factor | | |
| SASH3 | SAM and SH3 domain | 54440 | NM_018990.4 |
| | containing 3 | | |
| XBP1 | X-box binding protein 1 | 7494 | NM_001079539.1 |
| IL6 | Interleukin 6 | 3569 | NM_000600.5 |
| SLAMF6 | SLAM family member 6 | 114836 | NM_001184714.2 |
| BTK | Bruton tyrosine kinase | 695 | NM_000061.2 |
| ICAM1 | Intercellular adhesion | 3383 | NM_000201.3 |
| | molecule 1 | | |
| PARVG | Parvin gamma | 64098 | NM_001137605.2 |
| S100A9 | S100 calcium binding protein | 6280 | NM_002965.4 |
| | A9 | | |
| BTLA | B and T lymphocyte | 151888 | NM_001085357.2 |
| | associated | | |
| CD2 | CD2 molecule | 914 | NM_001328609.2 |
| TRAF3IP3 | TRAF3 interacting protein 3 | 80342 | NM_001287754.2 |
| MAP4K1 | Mitogen-activated protein | 11184 | NM_001042600.3 |
| | kinase kinase kinase kinase | | |
| | 1 | | |
| SLC31A1 | Solute carrier family 31 | 1317 | NM_001859.4 |
| | member 1 | | |
| TNFA | Tumor necrosis factor | 7124 | NM_000594.4 |
| KIAA0748 | Thymocyte expressed, | 9840 | NM_001098815.3 |
| | positive selection associated | | |
| | 1 | | |
| TIGIT | T cell immunoreceptor with | 201633 | NM_173799.4 |
| | Ig and ITIM domains | | |
| CXCR4 | C-X-C motif chemokine | 7852 | NM_001008540.2 |
| | receptor 4 | | |
| SP140 | SP140 nuclear body protein | 11262 | NM_001005176.3 |
| CD180 | CD180 molecule | 4064 | NM_005582.3 |
| ITGAX | Integrin subunit alpha X | 3687 | NM_000887.5 |
| IL21R | Interleukin 21 receptor | 50615 | NM_021798.4 |
| RAD51AP1 | RAD51 associated protein 1 | 10635 | NM_001130862.2 |
| DKK3 | Dickkopf WNT signaling | 27122 | NM_001018057.1 |
| | pathway inhibitor 3 | | |
| CSF1 | Colony stimulating factor 1 | 1435 | NM_000757.6 |
| TMEM43 | Transmembrane protein 43 | 79188 | NM_024334.2 |
| WNT11 | Wnt family member 11 | 7481 | NM_004626.3 |
| FGF9 | Fibroblast growth factor 9 | 2254 | NM_002010.3 |
| IL17D | Interleukin 17D | 53342 | NM_138284.1 |
| MXRA7 | Matrix remodeling associated | 439921 | NM_001008528.3 |
| | 7 | | |
| FMOD | Fibromodulin | 2331 | NM_002023.5 |
| PDGFA | Platelet derived growth factor | 5154 | NM_002607.5 |
| | subunit A | | |
| CDC14B | Cell division cycle 14B | 8555 | NM_001077181.3 |
| COMMD2 | COMM domain containing 2 | 51122 | NM_016094.4 |
| FKBP9 | FKBP prolyl isomerase 9 | 11328 | NM_001284341.1 |
| PTPRZ1 | Protein tyrosine phosphatase | 5803 | NM_001206838.2 |
| | receptor type Z1 | | |
| NOG | Noggin | 9241 | NM_005450.6 |
| SERTAD4 | SERTA domain containing 4 | 56256 | NM_001354173.2 |
| SNCAIP | Synuclein alpha interacting | 9627 | NM_001242935.2 |
| | protein | | |
| SLC37A3 | Solute carrier family 37 | 84255 | NM_001287498.1 |
| | member 3 | | |
| IGFBP6 | Insulin like growth factor | 3489 | NM_002178.3 |
| | binding protein 6 | | |
| LHFP | LHFPL tetraspan subfamily | 10186 | NM_005780.3 |
| | member 6 | | |
| CAV2 | Caveolin 2 | 858 | NM_001206747.2 |
| GK5 | Glycerol kinase 5 | 256356 | NM_001039547.3 |
| IL20 | Interleukin 20 | 50604 | NM_018724.3 |
| CILP | Cartilage intermediate layer | 8483 | NM_003613.4 |
| | protein | | |

Respective exemplary NCBI Gene ID and nucleic acid sequences (NCBI Accession No.) of further biomarkers of the invention include: IL8 (NCBI Gene ID 3576; exemplary NCBI Accession No. NM_000584.4), LTB (NCBI Gene ID 4050; exemplary NCBI Accession No. NM_002341.2), HIVEP1 (NCBI Gene ID 3096; exemplary NCBI Accession No. NM_002114.4), UBASH3A (NCBI Gene ID 53347; exemplary NCBI Accession No. NM_001001895.3) and IFNB1 (NCBI Gene ID 3456; exemplary NCBI Accession No. NM_002176.4).

In some embodiments, the one or more biomarkers are selected from Table 2 and the levels of the one or more biomarkers are increased compared to the corresponding reference values.

In some embodiments, the one or more biomarkers comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or all 49 biomarkers from Table 2.

In some embodiments, the one or more biomarkers comprise all 49 biomarkers from Table 2.

In some embodiments, the one or more biomarkers consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or all 49 biomarkers from Table 2.

In some embodiments, the one or more biomarkers consist of all 49 biomarkers from Table 2.

In some embodiments, the one or more biomarkers comprise one or more genes from Table 2 associated with B and T cell proliferation, differentiation and activation (e.g. TNFRSF13C, CD79A, CD2 and CD3E). In some embodiments, the one or more biomarkers comprise one or more biomarkers selected from TNFRSF13C, CD79A, CD2 and CD3E. In some embodiments, the one or more biomarkers comprise TNFRSF13C, CD79A, CD2 and CD3E. In some embodiments, the one or more biomarkers consist of one or more biomarkers selected from TNFRSF13C, CD79A, CD2 and CD3E. In some embodiments, the one or more biomarkers consist of TNFRSF13C, CD79A, CD2 and CD3E.

In some embodiments, the one or more biomarkers comprise one or more genes from Table 2 associated with matrix metallopeptidase production/regulation (e.g. MMP1). In some embodiments, the one or more biomarkers comprise MMP1. In some embodiments, the one or more biomarkers consist of MMP1.

In some embodiments, the one or more biomarkers comprise one or more genes from Table 2 associated with cytokine mediated cellular activation (e.g. TNFA and TRAF3IP3). In some embodiments, the one or more biomarkers comprise one or more biomarkers selected from TNFA and TRAF3IP3. In some embodiments, the one or more biomarkers comprise TNFA and TRAF3IP3. In some embodiments, the one or more biomarkers consist of one or more biomarkers selected from TNFA and TRAF3IP3. In some embodiments, the one or more biomarkers consist of TNFA and TRAF3IP3.

In some embodiments, the one or more biomarkers comprise one or more genes from Table 2 associated with osteoclastogenesis inhibition (e.g. DEF6). In some embodiments, the one or more biomarkers comprise DEF6. In some embodiments, the one or more biomarkers consist of DEF6.

TABLE 2
Biomarkers of Table 1 that are upregulated
Gene name
GPR114
CCL19
MMP3
MMP1
CXCL13
CD79A
TNFRSF17
IGJ
ZBP1
SLAMF7
PIM2
CXCL1
FCRL5
MS4A1
PLA2G2D
KIAA0125
CD19
MMP10
CD3E
IL2RG
SEL1L3
KCNA3
TNFRSF13C
SIRPG
TMEM156
CD40LG
DEF6
SASH3
XBP1
IL6
SLAMF6
BTK
ICAM1
PARVG
S100A9
BTLA
CD2
TRAF3IP3
MAP4K1
SLC31A1
TNFA
KIAA0748
TIGIT
CXCR4
SP140
CD180
ITGAX
IL21R
RAD51AP1

In some embodiments, the one or more biomarkers are selected from Table 3 and the levels of the one or more biomarkers are decreased compared to the corresponding reference values.

In some embodiments, the one or more biomarkers comprise at least 2, 3, 4, 5, 6, 7, 8, 9, 10, 11, 12, 13, 14, 15, 16, 17, 18, 19, 20, 21, 22 or all 23 biomarkers from Table 3.

In some embodiments, the one or more biomarkers comprise all 23 biomarkers from Table 3.

In some embodiments, the one or more biomarkers consist of 2, 3, 4, 5, 6, 7, 8, 9, 10, 11, 12, 13, 14, 15, 16, 17, 18, 19, 20, 21, 22 or all 23 biomarkers from Table 3.

In some embodiments, the one or more biomarkers consist of all 23 biomarkers from Table 3.

TABLE 3
Biomarkers of Table 1 that are downregulated
Gene name
DKK3
CSF1
TMEM43
WNT11
FGF9
IL17D
MXRA7
FMOD
PDGFA
CDC14B
COMMD2
FKBP9
PTPRZ1
NOG
SERTAD4
SNCAIP
SLC37A3
IGFBP6
LHFP
CAV2
GK5
IL20
CILP

The increase in the level of the one or more biomarker compared to the corresponding reference values may, for example, be an increase in the level of at least about 10%, 20%, 30%, 40%, 50%, 60%, 70%, 80%, 90%, 95%, 96%, 97%, 98% or 99% or greater relative to the reference value. The increase in the level of the one or more biomarker compared to the corresponding reference values may, for example, be an increase in the level of at least about 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.5×, 4×, 4.5×, 5×, 6×, 7×, 8×, 9×, 10×, 15×, 20×, 30×, 40×, 50×, 100×, 500× or 1000× relative to the reference value.

The decrease in the level of the one or more biomarker compared to the corresponding reference values may, for example, be a decrease in the level of at least about 10%, 20%, 30%, 40%, 50%, 60%, 70%, 80%, 90%, 95%, 96%, 97%, 98% or 99% or greater relative to the reference value.

In some embodiments, the one or more biomarkers does not comprise a biomarker selected from the group consisting of CCL19, MMP1, TNFRSF17, PIM2, CXCL1, FCRL5, CD19, MMP10, SEL1L3, SIRPG, CD40LG, XBP1, SLAMF6, BTK, BTLA, TRAF3IP3, MAP4K1, SLC31A1, TNFA, TIGIT, CD180, DKK3, FGF9, NOG and CILP.

In some embodiments, the one or more biomarkers does not comprise CCL19. In some embodiments, the one or more biomarkers does not comprise MMP1. In some embodiments, the one or more biomarkers does not comprise TNFRSF17. In some embodiments, the one or more biomarkers does not comprise PIM2. In some embodiments, the one or more biomarkers does not comprise CXCL1. In some embodiments, the one or more biomarkers does not comprise FCRL5. In some embodiments, the one or more biomarkers does not comprise CD19. In some embodiments, the one or more biomarkers does not comprise MMP10. In some embodiments, the one or more biomarkers does not comprise SEL1 L3. In some embodiments, the one or more biomarkers does not comprise SIRPG. In some embodiments, the one or more biomarkers does not comprise CD40LG. In some embodiments, the one or more biomarkers does not comprise XBP1. In some embodiments, the one or more biomarkers does not comprise SLAMF6. In some embodiments, the one or more biomarkers does not comprise BTK. In some embodiments, the one or more biomarkers does not comprise BTLA. In some embodiments, the one or more biomarkers does not comprise TRAF3IP3. In some embodiments, the one or more biomarkers does not comprise MAP4K1. In some embodiments, the one or more biomarkers does not comprise SLC31A1. In some embodiments, the one or more biomarkers does not comprise TNFA. In some embodiments, the one or more biomarkers does not comprise TIGIT. In some embodiments, the one or more biomarkers does not comprise CD180. In some embodiments, the one or more biomarkers does not comprise DKK3. In some embodiments, the one or more biomarkers does not comprise FGF9. In some embodiments, the one or more biomarkers does not comprise NOG. In some embodiments, the one or more biomarkers does not comprise CILP.

In some embodiments, the one or more biomarkers does not comprise any of CCL19, MMP1, TNFRSF17, PIM2, CXCL1, FCRL5, CD19, MMP10, SEL1L3, SIRPG, CD40LG, XBP1, SLAMF6, BTK, BTLA, TRAF3IP3, MAP4K1, SLC31A1, TNFA, TIGIT, CD180, DKK3, FGF9, NOG and CILP.

Additional Clinical Covariates

The methods disclosed herein may further comprise determining one or more clinical covariates of the subject. Alternatively or additionally, one or more clinical covariates may have been determined for the subject. The method may comprise comparing the one or more clinical covariates to one or more reference values. Example clinical covariates include Disease Activity Score (DAS), DAS28, baseline pathotype, C-reactive protein and tender joint count (TJC).

In some embodiments, the one or more clinical covariates are selected from the group consisting of Disease Activity Score (DAS), DAS28, baseline pathotype, C-reactive protein and tender joint count (TJC).

In some embodiments, the method further comprises the step of determining the baseline pathotype of the subject. In some embodiments, the baseline pathotype has been determined for the subject. In some embodiments, the method further comprises the step of determining whether the subject exhibits a lympho-myeloid pathotype.

In some embodiments, a lympho-myeloid pathotype is indicative of the requirement for treatment with a biologic therapy for rheumatoid arthritis.

The term “pathotype” as used herein may refer to a subtype of RA characterised by pathological, histological and/or clinical features of RA. Such pathotypes include, but are not limited to, the lymphoid pathotype (e.g. characterised by B cell-rich aggregates), myeloid pathotype (e.g. characterised by a predominant macrophage infiltrate) and pauciimmune-fibroid pathotype (e.g. characterised by and few infiltrating immune cells, but still expansion of fibroblast lineage cells in the sublining and lining layers).

Determining the Level of One or More Biomarkers

Methods for determining biomarker levels are well known in the art and would be familiar to the skilled person.

For example, the level of a biomarker may be determined by measuring gene expression for the biomarker gene (for example, using RTPCR) or by detecting the protein product of the biomarker gene (for example, using an immunoassay).

In some embodiments, the step of determining the levels of the one or more biomarkers comprises determining the levels of gene expression of the one or more biomarkers.

In some embodiments, the level is a nucleic acid level. In some embodiments, the nucleic acid level is an mRNA level.

In some embodiments, the level of the one or more biomarkers is determined by direct digital counting of nucleic acids (e.g. by Nanostring, for example as disclosed in the Examples herein), RNA-seq, RT-qPCR, qPCR, multiplex qPCR or RT-qPCR, microarray analysis, or a combination thereof.

In some embodiments, the level is a protein level.

In some embodiments, the level of the one or more biomarkers is determined by an immunoassay, liquid chromatography-mass spectrometry (LC-MS), nephelometry, aptamer technology, or a combination thereof.

In some embodiments, the level of the one or more biomarkers is an average of the level of the one or more biomarkers. In some embodiments, the average of the level of the one or more biomarkers is an average of a normalised level of the one or more biomarkers.

In some embodiments, the level of the one or more biomarkers is a median of the level of the one or more biomarkers. In some embodiments, the median of the level of the one or more biomarkers is a median of a normalised level of the one or more biomarkers.

In some embodiments, the level of the one or more biomarkers is the level of the one or more biomarkers normalised to a reference gene (e.g. ACTB, GAPDH, GUSB, HPRT 1, PGK1, RPL19, TUBB, TMEM55B or a combination thereof).

Sample

The method of the invention is carried out on one or more samples obtained from a subject, for example a patient suspected of having RA.

Samples may be obtained from a joint of a subject, for example from a biopsy. Samples may be obtained from a synovial tissue sample from a subject.

As used herein, the term “synovial sample” refers to a sample derived from a synovial joint. Typically, the synovial sample will be derived from a synovial joint of a RA patient. A synovial sample may be a synovial tissue biopsy and the synovial joint may display active inflammation at the time the sample is taken.

Methods for obtaining samples, such as synovial tissue samples are well known in the art and would be familiar to the skilled person. For example, techniques such as ultrasound (US)-guided biopsies may be used to obtain tissue samples.

In some embodiments, the sample is a synovial sample. In some embodiments, the sample is a synovial tissue sample or a synovial fluid sample.

In some embodiments, the sample is obtained by synovial biopsy, preferably ultrasound-guided synovial biopsy.

Reference Values

The method of the invention comprises the step of comparing the level of one or more biomarkers to one or more corresponding reference values.

As used herein, the term “reference value” may refer to an expression level against which another expression level (e.g. the level of one or more biomarkers disclosed herein) is compared (e.g. to make a diagnostic (e.g. predictive and/or prognostic) and/or therapeutic determination).

For example, the reference value may be derived from expression levels in a reference population (e.g. the median expression level in a reference population), for example a population of patients having RA who have not been treated with an RA therapy; a reference sample; and/or a pre-assigned value (e.g. a cut-off value which was previously determined to significantly separate a first subset of individuals who required biologic therapy for rheumatoid arthritis and a second subset of individuals who did not).

In some embodiments, the cut-off value may be the median or mean expression level in the reference population. In some embodiments, the reference level may be the top 40%, the top 30%, the top 20%, the top 10%, the top 5% or the top 1% of the expression level in the reference population.

A corresponding reference value may be derived from a subject without RA, for example a subject with osteoarthritis (OA).

The reference value may, for example, be based on a mean or median level of the biomarker in a control population of subjects, e.g. 5, 10, 100, 1000 or more subjects (who may be age- and/or gender-matched, or unmatched to the test subject).

In certain embodiments the reference value may have been previously determined, or may be calculated or extrapolated without having to perform a corresponding determination on a control sample with respect to each test sample obtained.

Subject

In preferred embodiments, the subject is a human.

In preferred embodiments the subject is an adult human. In some embodiments, the subject may be a child or an infant.

In preferred embodiments, the subject has not been previously treated for rheumatoid arthritis. Preferably, the subject is treatment naïve for Disease-Modifying Anti-Rheumatic Drugs (DMARDs) and/or steroids.

In some embodiments, the subject has not been previously treated with a Disease-Modifying Anti-Rheumatic Drug (DMARD). In some embodiments, the subject has not been previously treated with a biologic therapy for rheumatoid arthritis. In preferred embodiments, the subject has not been previously treated with a Disease-Modifying Anti-Rheumatic Drug (DMARD) or a biologic therapy for rheumatoid arthritis.

In some embodiments, the subject is suspected of having rheumatoid arthritis. In some embodiments, the subject presents one or more symptoms associated with RA. In some embodiments, has been diagnosed with rheumatoid arthritis (RA).

In some embodiments, the subject has presented one or more symptoms of rheumatoid arthritis for less than 1 year, for example less than 11, 10, 9, 8, 7, 6, 5, 4 or 3 months.

Antibodies

The term “antibody” is used herein to relate to an antibody or a functional fragment thereof. By functional fragment, it is meant any portion of an antibody which retains the ability to bind to the same antigen target as the parental antibody.

As used herein, “antibody” means a polypeptide having an antigen binding site which comprises at least one complementarity determining region (CDR). The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′2, Fv, single chain Fv (ScFv) fragment or Nanobody. The antibody may be a conjugate of the antibody and another agent or antibody, for example the antibody may be conjugated to a polymer (e.g. PEG), toxin or label. The antibody may be a bifunctional antibody. The antibody may be non-human, chimeric, humanised or fully human.

Methods of Treatment

The invention also provides a method for treating a subject for rheumatoid arthritis, the method comprising administering to the subject an effective amount of a biologic therapy for rheumatoid arthritis, wherein the subject has been identified as requiring treatment with a biologic therapy for rheumatoid arthritis by the method of the invention as disclosed herein.

The biologic therapy for rheumatoid arthritis may be biologic therapy as disclosed herein.

Kits

The present invention also provides a kit suitable for performing the method as disclosed herein. In particular, the kit may comprise reagents suitable for detecting the biomarkers disclosed herein, or a biomarker combination as disclosed herein.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

Methods

Patients

200 consecutive inflammatory arthritis patients recruited at Barts Health NHS Trust as part of the multi-centre pathobiology of early arthritis cohort (http://www.peac-mrc.mds.qmul.ac.uk) were included within the study. Patients were treatment naïve (csDMARD and steroid) and had <1 year symptoms.

At baseline patients underwent collection of routine demographic data and were categorised according to the following criteria: (i) RA1987 (Arnett F C et al. (1987) THE AMERICAN RHEUMATISM ASSOCIATION 1987 REVISED CRITERIA FOR THE CLASSIFICATION OF RHEUMATOID ARTHRITIS) or (ii) UA. 2010 ACR/EULAR criteria for RA (Aletaha D et al. (2010) Rheumatoid arthritis classification criteria: an American College of Rheumatology/European League Against Rheumatism collaborative initiative 1580-8) were then applied to further classify patients with UA, resulting in three groups: (i) RA1987 (RA1987+/RA2010+), (ii) RA2010 (RA1987−/RA2010+) and (iii) UA (RA1987−/RA2010−). An ultrasound (US) guided synovial biopsy of a clinically active joint was performed (Kelly S et al. (2013) Ann Rheum Dis 74: 611-7). Patients were then commenced on standard conventional synthetic (cs)DMARD therapy with a treat-to-target approach to treatment escalation (DAS28<3.2). Patients failing csDMARD therapy were commenced on biologic therapy (anti-TNF, Tocilizumab or Rituximab) according to the prevailing UK National Institute for Clinical Excellence (NICE) prescribing algorithm if they continued to have a DAS28>5.1 following 6 months of therapy (Overview|Rheumatoid arthritis in adults: management|Guidance|NICE. https://www.nice.org.uk/guidance/ng100 (accessed 2 Jul. 2019)). At 12 months follow-up patients were categorised as follows: i. self-limiting (SL) disease (DAS28<3.2 and off csDMARD/steroid therapy) vs persistent disease (PD) (DAS28>3.2 and/or csDMARD) and ii. Symptomatic treatment (non-steroidal anti-inflammatories) treatment vs csDMARD therapy vs Biologic+/−csDMARD therapy.

Synovial Biopsy Collection and Processing

A minimum of 6 biopsies per patient were collected for paraffin embedding and if intact lining layer identified underwent histopathological assessment. Synovitis score was determined using a previously validated scoring system (Krenn V et al. (2006) Histopathology 49: 358-64). Following immunohistochemical staining of sequentially cut slides using previously reported protocols for B cells (CD20), T cells (CD3), macrophages (CD68) and plasma cells (CD138) the degree of immune cell infiltration was assessed semi-quantitatively (0-4) (Humby F et al. (2009) PLoS Med 6: 0059-75). Biopsies were stratified into 1 of 3 synovial pathotypes according to the following criteria: i) Lympho-myeloid presence of grade 2-3 CD20+aggregates, (CD20≥2) and/or CD138>2 ii) diffuse-myeloid CD68 SL≥2, CD20≤1 and/or CD3≥1, CD138≤2 and iii) pauciimmune CD68 SL<2 and CD3, CD20, CD138<1

Nanostring Analysis

A minimum of 6 synovial samples per patient were immediately immersed in RNA-Later and RNA extraction performed as described (Humby F et al. (2019) Ann Rheum Dis annrheumdis-2018-214539, doi:10.1136/annrheumdis-2018-214539). RNA samples then underwent profiling for expression of 238 genes preselected based on previous microarray analyses of synovial tissue from patients with established RA (Dennis G et al. (2014) Arthritis Res Ther 16: R90) and/or relevance to RA pathogenesis. Raw NanoString counts were processed using the NanoStringQCPro package in R 3.2.0. Counts were normalised for RNA content by global gene count normalisation and then log transformed (base 2). The validity of normalisation was then checked via box- and scatter plots of normalised counts. Benjamini-Hochberg method was used to adjust for multiple testing, and genes were considered to be differentially expressed if they demonstrated an FDR-adjusted p-value <0.01.

Statistical Analysis

Statistical analyses were run using R.3.0.2. For three way comparisons, Kruskal-Wallis test was used for continuous and Chi-squared or Fisher's exact test used for categorical variables as appropriate. A p-value <0.05 was considered statistically significant. Post hoc comparison tests were performed using Dunn test or Bonferroni correction as appropriate.

Linear regression models: Logistic regression using forward, backward and bidirectional stepwise selection was employed using the glm function in R.

Gene expression predictors were selected by L1 (LASSO) sparse logistic regression using R package glmnet. The penalty parameter λ was optimised using 10-fold cross-validation. A corresponding to the minimum mean cross-validated error was retained as final penalty parameter in the model.

Predictive performance evaluation: Predictive performance of the final prediction model was assessed by computing the area under the receiver operating characteristic curve (AUC), using both apparent and internal validation with 95% CI. Internal validation using a bootstrap method (Smith G C S et al. (2014) Am J Epidemiol 180: 318-24; Efron B et al. An introduction to the bootstrap. Chapman & Hall 1994. https://www.crcpress.com/An-Introduction-to-the-Bootstrap/Efron-Tibshirani/p/book/9780412042317 (accessed 27 Feb. 2019)) (performed with R package boot version 1.3-18) was employed to correct for over-fitting, to generate unbiased optimism-adjusted estimates of the C statistic (AUC) with low absolute error.

Bootstrap estimate of the AUC statistic was computed by random sampling with replacement 500 times to enable estimation of the optimism corrected AUC.

Results

Patient Demographics and Clinical Correlations

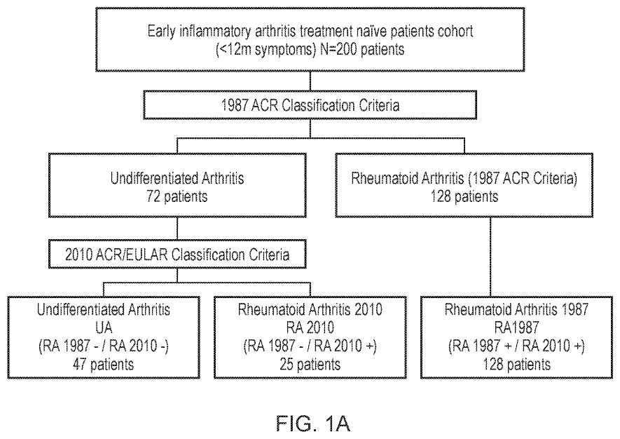

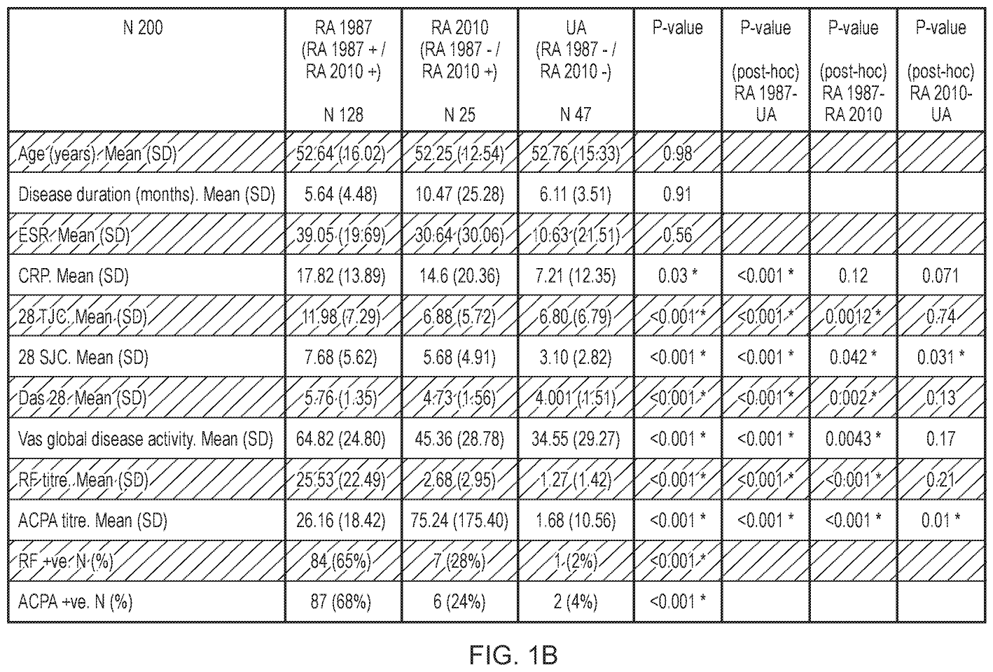

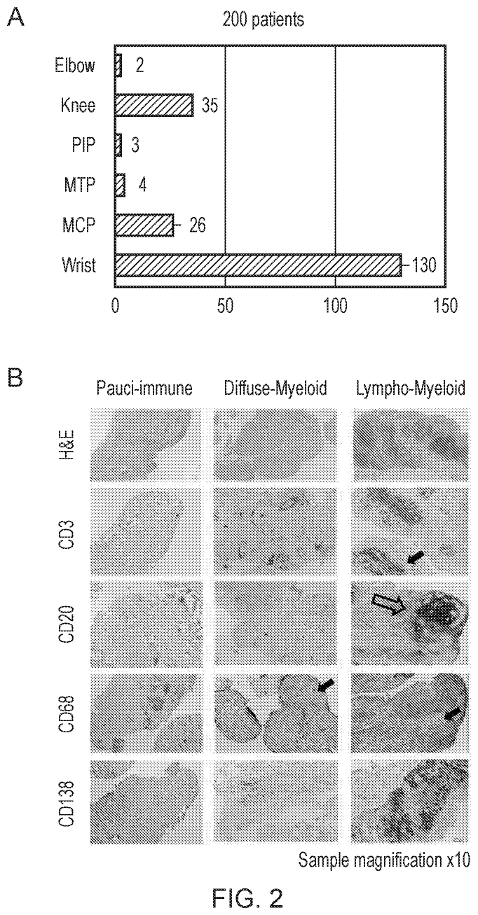

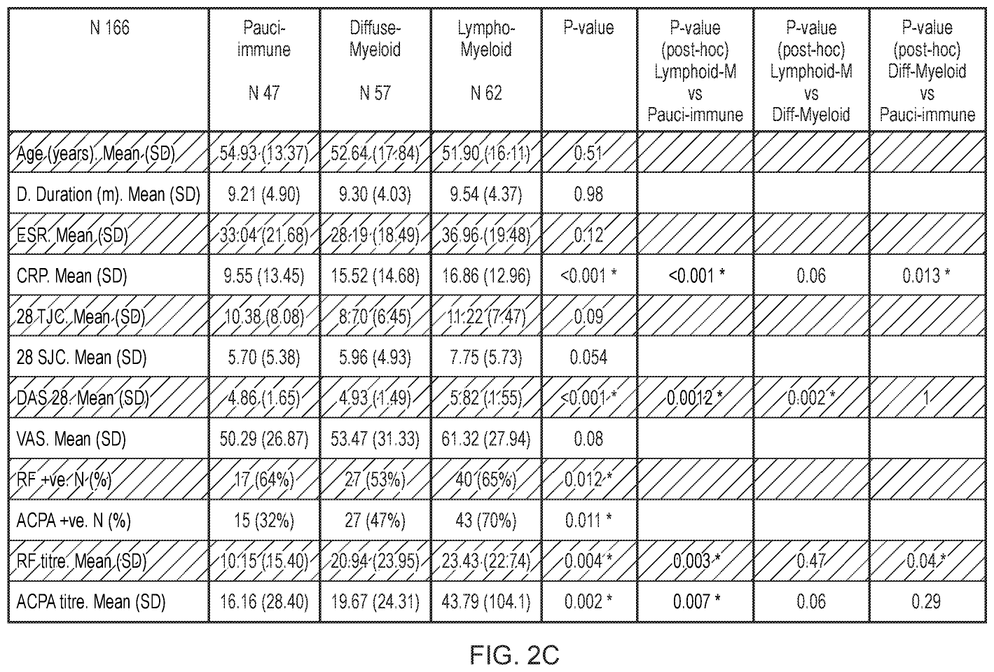

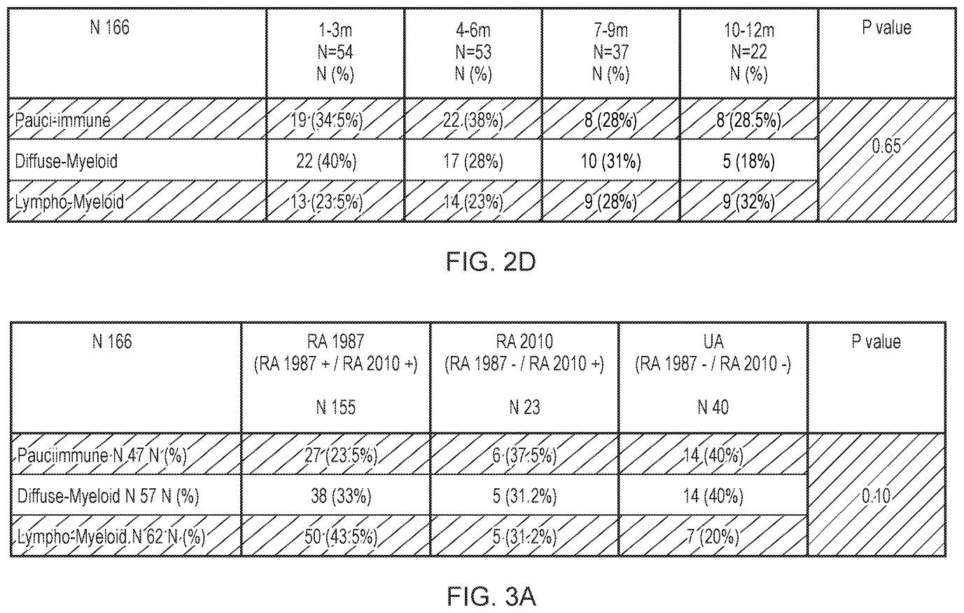

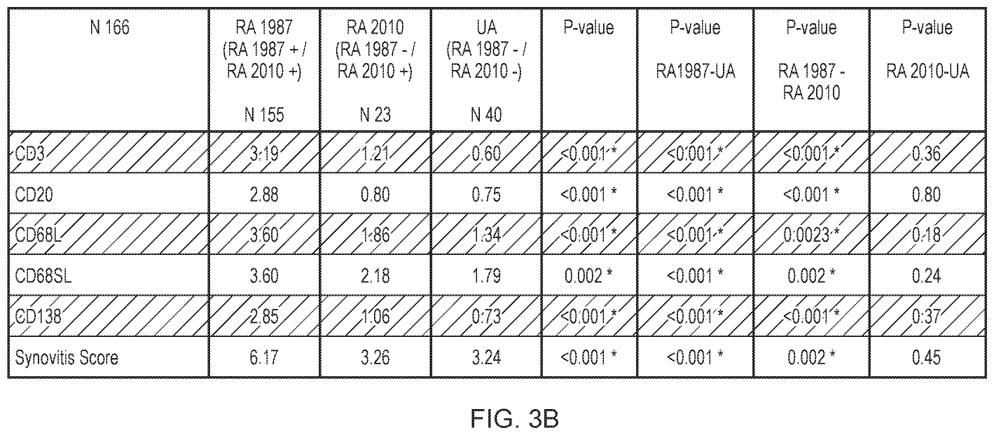

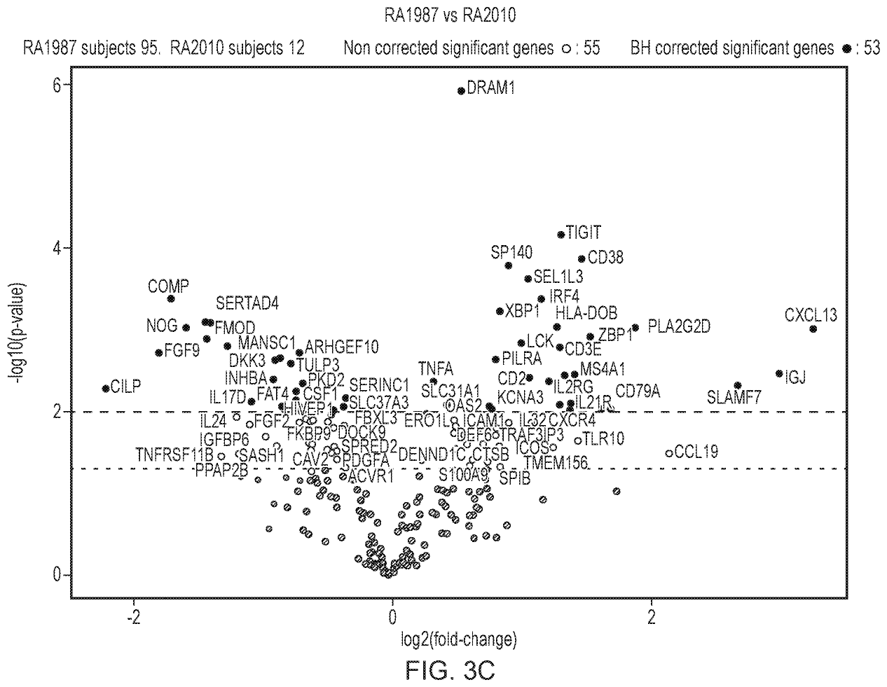

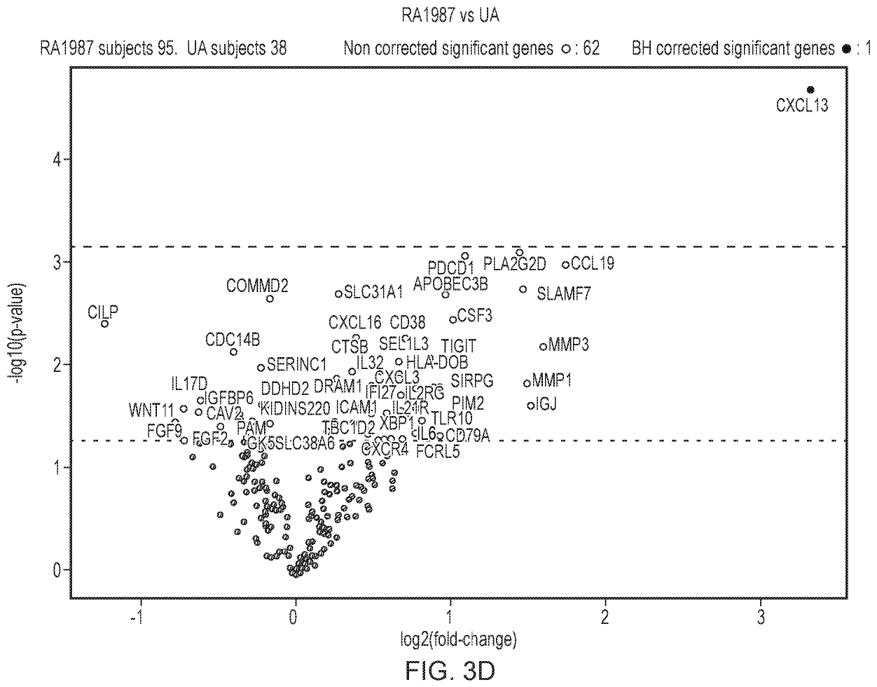

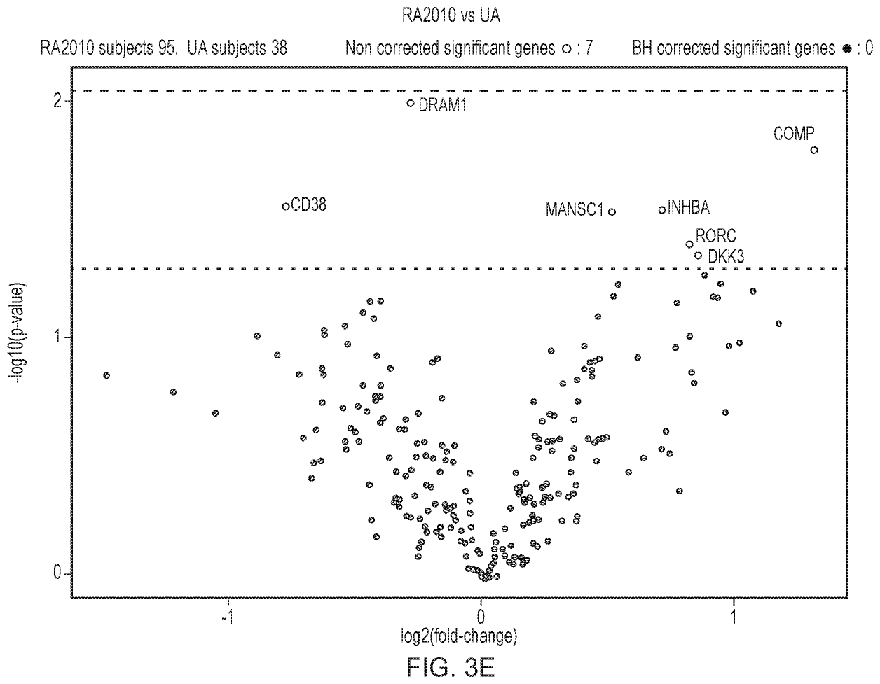

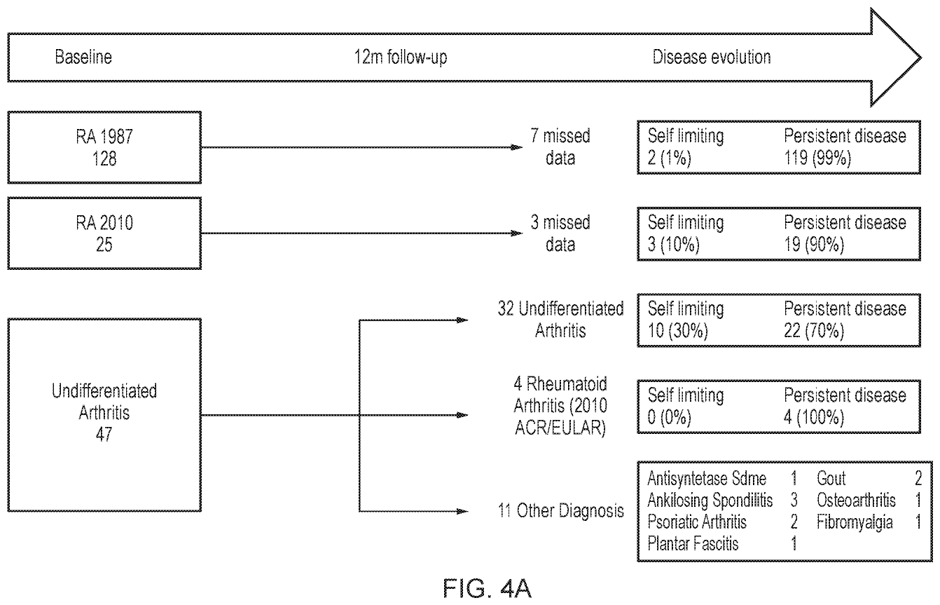

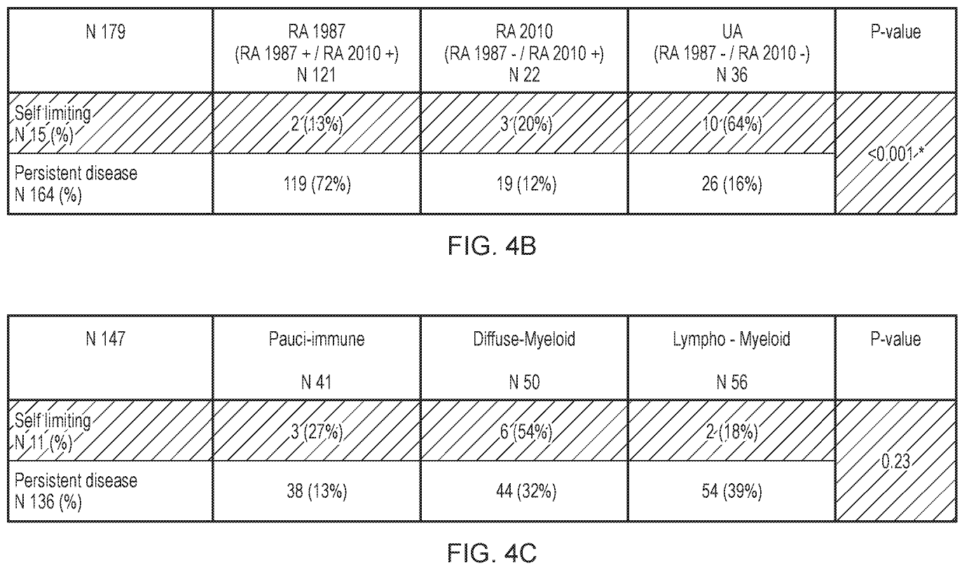

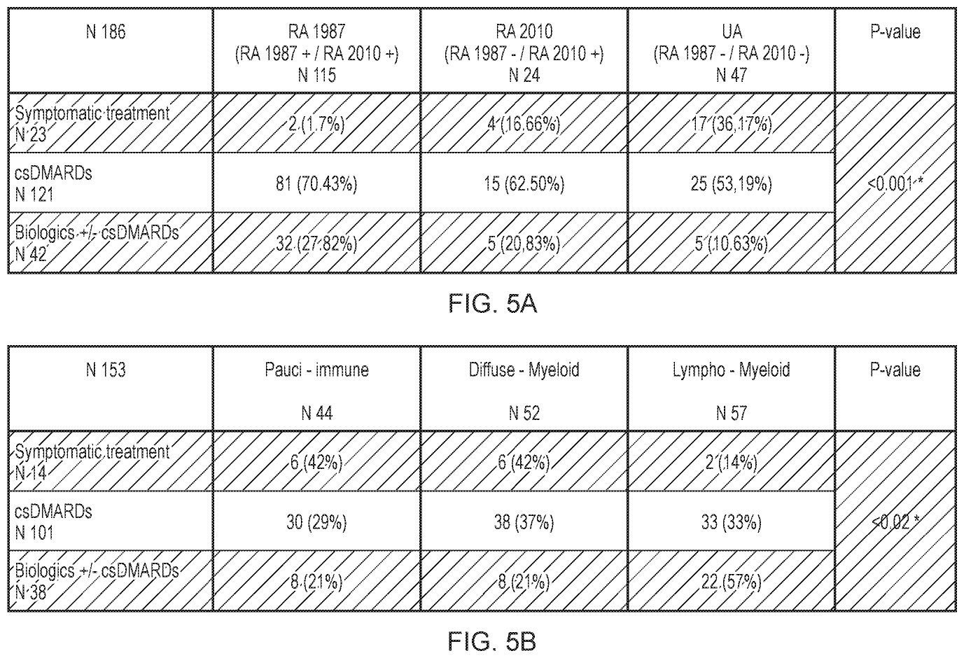

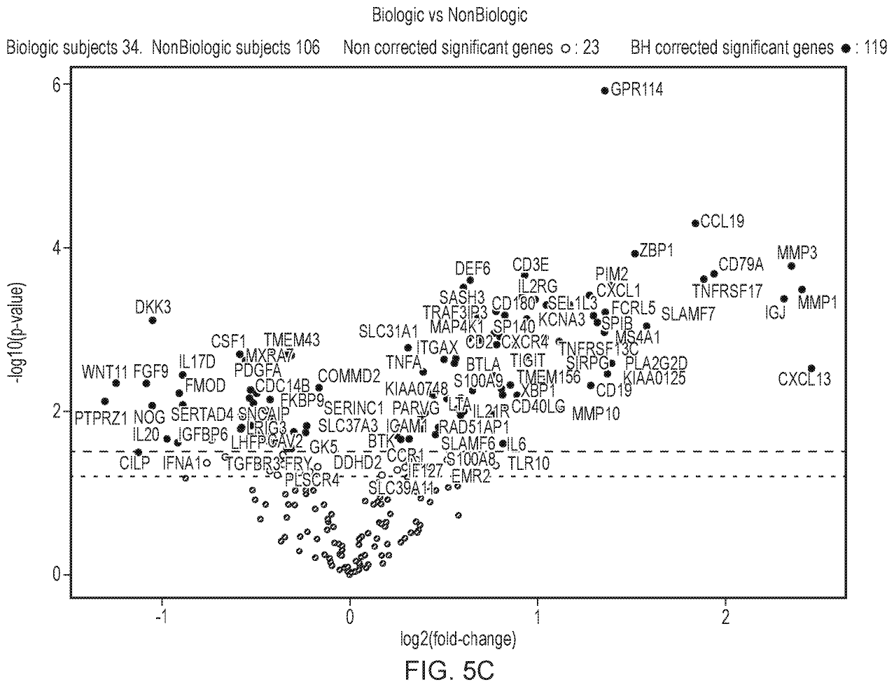

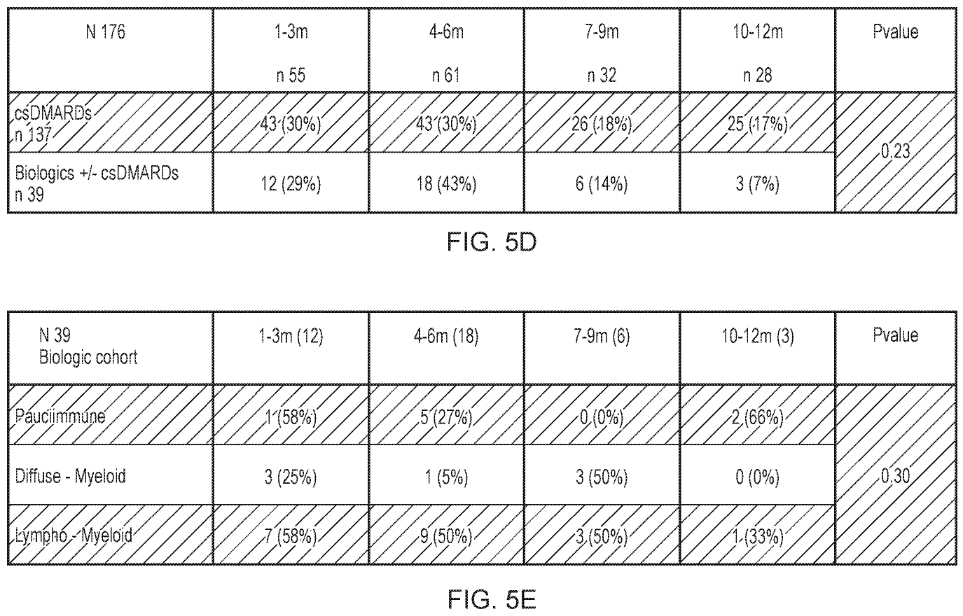

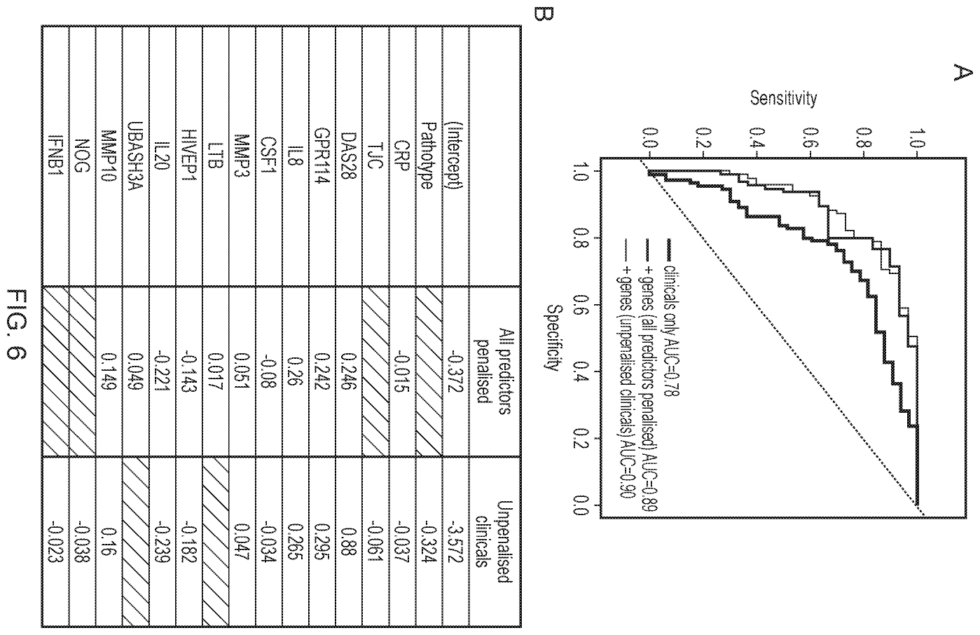

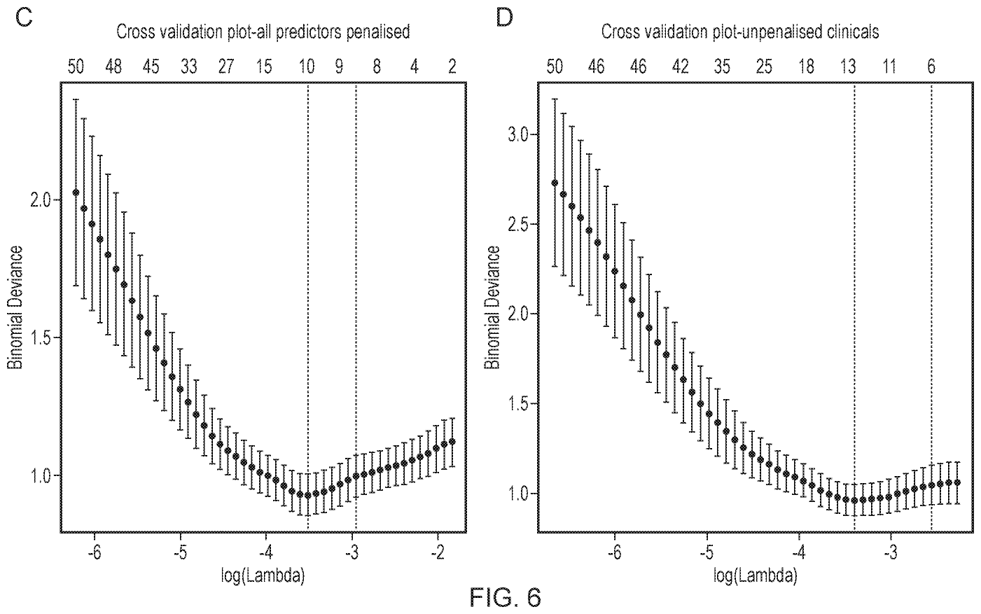

200 PEAC patients were included, 128/200 (64%) patients were classified as RA1987 (RA 1987+/RA2010+) and 72/200 (36%) as UA. Of the UA patients, 25 were further classified as RA2010 (RA1987−/RA2010+) (25/200, 12.5%) and 47 remained as UA (RA1987−/RA2010−) (47/200, 23.5%) (FIG. 1A). No significant difference in mean age, disease duration or ESR between groups was demonstrated. However, the RA1987 group had significantly higher levels of CRP, TJC, SJC, DAS28, RF, ACPA and VAS and significantly higher numbers of patients sero positive for RF and ACPA compared to either the RA2010 or UA groups (FIG. 1B). SJC and ACPA titre were the only clinical parameters with significant differences between the RA2010 and UA groups, indicating that in terms of clinical measures of disease activity these two groups are relatively homogenous.

Synovial Pathotypes Distinguish Clinical Phenotypes Regardless of Disease Duration

Synovial biopsies were obtained predominantly from small joints (81.5%) (FIG. 2A). Patients with synovial tissue suitable for histological analysis (166/200) were segregated according to baseline synovial pathotype (FIG. 2B) and differences in clinical parameters evaluated. We demonstrated significantly higher mean DAS28 within the lympho-myeloid compared to either the diffuse-myeloid or pauciimmune group (5.82 vs 4.93 vs 4.86, p<0.001). Mean CRP was significantly higher in the lympho-myeloid and diffuse-myeloid vs pauciimmune groups (16.86 vs 15.52 vs 9.55, p<0.001) and a significantly higher number of patients were sero-positive for either RF (p=0.012) or ACPA (p=0.011) within the lympho-myeloid group (FIG. 2C). To evaluate whether disease duration influenced prevalence of synovial pathotype, patients were stratified into four groups according to disease duration at baseline (1-3 m, 4-6 m, 7-9 m and 10-12 m) and frequency of synovial pathotype determined. No significant differences in synovial pathotype frequency at each time point was demonstrated (p=0.65) (FIG. 2D).

RA1987 Patients Display Significantly Higher Levels of Synovial Immune Cell Infiltration Compared to RA2010 and UA Patients

Patients were segregated according to pathotype and further into RA1987, RA2010 and UA categories. A higher proportion of patients within the RA1987 group were categorised as lympho-myeloid (vs diffuse-myeloid or pauciimmune) (43.5% vs 33% vs 23.5%) (FIG. 3A). We also demonstrated a significantly higher mean synovitis, CD3+ T cell, CD20+ B cell, CD138+ plasma cell and CD68+ SL/L macrophage score between the RA1987 group and both the RA2010 and UA groups (p<0.001) (FIG. 3B). We saw no significant differences in synovitis score, mean CD3+T, CD20+B, CD68+ L or SL macrophage or CD138+ plasma cell number between the RA2010 and UA group (FIG. 3B), indicating that these two groups are relatively homogenous in terms of tissue pathology.

Synovial Genes Regulating B Cell Activation and Function are Significantly Upregulated in RA1987 Patients Compared to the RA2010/UA Groups.

145/200 patients had RNA available for nanostring analysis (95/128 RA1987, 12/25 RA2010 and 38/47 UA patients) and were analysed for differential gene expression (238 genes) between groups.

Comparing RA1987 vs RA2010 groups we demonstrated a significant differential expression of 53 genes (FIG. 3C). In line with the histological analysis a number of differentially upregulated genes within the RA1987 cohort were involved in mediating B cell activation/function (e.g. CD79A, CD38, IGJ, CXCL13, IRF4, CCL19, CD38, TNFA, and IL6). When evaluating gene expression between RA1987 and UA groups we found a similar trend with differential upregulation of a number of genes within the RA1987 cohort mediating B cell activation/function although only CXCL13 remained significant following correction for multiple comparisons (FIG. 3D). Conversely when evaluating gene expression between the RA2010 and UA cohorts only 7 genes appeared as significant with a preponderance of differentially upregulated genes within the RA2010 cohort mediating cartilage biology (COMP, DKK3, INHBA) and none remaining significant after correction for multiple comparisons (FIG. 3E).

Classification as RA1987 Criteria at Disease Onset Predicts Persistent Disease at 12 Months

190/200 patients had 12 month follow up data available, we examined whether baseline synovial pathotype was associated with disease evolution. 119/121 (99%) RA1987 patients and 19/22 (90%) RA2010 had PD (FIG. 4A). Within the UA cohort 11/47 (23%) had other diagnoses. Of the remaining 36 patients, 26/36 (72.2%) had PD, and 10/36 (27.8%) SL. Of the UA patients with PD 4/26 (15.3%) progressed to fulfil 2010ACR/EULAR criteria RA at 12 months. Results demonstrated a significantly higher proportion of patients with SL disease in the UA group compared to the RA2010 or RA1987 groups and a significantly higher number of patients within the RA1987 group with PD (FIG. 4B). When evaluating the effect of baseline pathotype we demonstrated a higher proportion of patients with a lympho-myeloid vs diffuse-myeloid or pauciimune pathotype (39% vs 32% vs 13%) with PD and a higher number of patients with a diffuse-myeloid vs lympho-myeloid or pauciimmune pathotype (54% vs 18% vs 27%) with SL (FIG. 4C).

A Baseline Lympho-Myeloid Pathotype Significantly Associates with 12 Month Requirement for Biologic Therapy.

Patients stratified according to diagnostic group or pathotype were further classified according to 12 month treatment requirement: i. symptomatic treatment, ii. csDMARDs or iii. biologics+/−csDMARDs. A significantly higher proportion of RA1987 patients required biologic compared with RA2010 and UA (27.82% vs 20.83% vs 10.63%) (p<0.001) (FIG. 5A) and importantly, lympho-myeloid (vs diffuse-myeloid or pauciimmune) pathotype significantly associated with 12 month requirement for biologic therapy (57% vs 21% vs 21% p=0.02) (FIG. 5B).

We then compared expression of the 238 genes in the Nanostring panel between patients requiring biologic therapy (n=34) or not (n=106) and found 119 differentially expressed genes. Patients requiring biologic therapy had significantly higher differential upregulation of genes regulating B and T cell proliferation, differentiation and activation (e.g. TNFRSF13C, CD79A, CD2, CD3E and CD38), genes involved in matrix metallopeptidase production/regulation (e.g. MMP1 and TIMP1), genes involved in cytokine mediated cellular activation (TNFA, TRAF3P3, IFNA1), and osteoclastogenesis inhibition (DEF6). Patients who did not require biologic therapy expressed some B and T cell regulation genes and B proliferation markers but mostly markers of fibroblast proliferation and cartilage turnover (FIG. 5C).

To determine whether disease duration influenced outcome we segregated patients according to 12 month treatment (biologic therapy or not) and further into disease duration quartiles (FIG. 5D) and demonstrated no significant differences in terms of disease duration at diagnosis. Next, we segregated patients treated with biologic therapy (n=39) according to quartiles of disease duration and then synovial pathotype. We found no significant differences in patient number in each quartile (P=0.3) (FIG. 5E). These results strongly suggest that synovial pathotype rather than disease duration influences 12 month treatment outcome.

Synovial Gene Expression Signatures Enhance the Performance of Clinical Prediction Models for Biologic Requirement

To determine whether baseline clinical and gene expression data could be combined into a model for predicting requirement for biologic therapy, we used 2 complementary approaches: a logistic regression model to identify predictive clinical covariates, and a penalised method based on logistic regression with an L1 regularisation penalty (LASSO) to identify genes improving the clinical model.

9 baseline clinical covariates were considered as candidates in the regression model: disease duration, ESR, CRP, RF, ACPA, TJC, SJC, DAS28, and pathotype (two categories, lympho-myeloid vs pauciimmune/diffuse-myeloid). Logistic regression models using backward forward and bidirectional stepwise selection resulted in selection of the same set of clinical covariates: DAS28, pathotype, CRP and TJC. The apparent predictive performance of the model evaluated by AUC was 0.78 (95% CI=0.70-0.87).

Genes were selected to improve the clinical model using logistic regression with an L1 regularisation penalty (LASSO) applied on the 4 clinical covariates selected by the previous logistic regression and the 119 genes identified as being significantly differentially expressed between the biologic and non-biologic groups. Models in which clinical predictors were penalised or subject to forced inclusion were compared. When all predictors were penalised, 11 predictors were retained in the final model and when the clinical covariates were not penalised, 13 predictors were retained (FIG. 6A). In both the penalised and unpenalised clinical model the apparent prediction performance was improved (apparent AUC=0.89, 95% CI=0.83-0.95 and AUC=0.90, 95% CI=0.84-0.95) (FIG. 6B). We additionally performed internal validation to correct the AUC performance measure for over-fitting by calculating the optimism of the AUC for each model by boot-strapped sampling with replacement from the original dataset. The optimism corrected AUC was 0.75 for the pure clinical model and 0.81 for the clinical and gene model (LASSO) (FIGS. 6C and 6D) suggesting that including both clinical covariates and genes in the model results in an improvement of the predictive ability of the model.

The genes used in the model are shown in the table below.

TABLE 4
Significant differential regulation of gene expression
(Biologic vs No Biologic Treatment at 12m).

| Down regulation | Upregulation | |
|---|---|---|
| DKK3 | GPR114 | PARVG |
| CSF1 | CCL19 | S100A9 |
| TMEM43 | MMP3 | BTLA |
| WNT11 | MMP1 | CD2 |
| FGF9 | CXCL13 | DEF6 |
| IL17D | CD79A | SASH3 |
| TMEM43 | TNFRSF17 | TRAF3IP3 |
| MXRA7 | IGJ | MAP4K1 |
| FMOD | ZBP1 | SLC31A1 |
| PDGFA | SLAMF7 | TNFA |
| CDC14B | PIM2 | KIAA0748 |
| COMMD2 | CXCL1 | BTLA |
| FKBP9 | FCRL5 | TIGIT |
| PTPRZ1 | MS4A1 | CXCR4 |
| NOG | PLA2G2D | SP140 |
| SERTAD4 | KIAA0125 | CD180 |
| SNCAIP | CD19 | ITGAX |
| SLC37A3 | MMP10 | XBP1 |
| IGFBP6 | CD3E | CD40LG |
| LHFP | IL2RG | IL21R |
| CAV2 | SEL1L3 | RAD51AP1 |
| GK5 | KCNA3 | PARVG |
| IL20 | TNFRSF13C | SLC31A1 |
| CILP | SIRPG | ITGAX |
| | TMEM156 | CD2 |
| | CD40LG | |
| | DEF6 | |
| | SASH3 | |
| | XBP1 | |
| | IL6 | |
| | SLAMF6 | |
| | BTK | |
| | ICAM1 | |

Discussion

These results strongly suggest that early inflammatory arthritis patients not fulfilling RA1987 criteria display similar clinical, synovial histological and molecular features irrespective of further classification according to RA2010 or UA criteria. These data also suggest that a lympho-myeloid pathotype at disease onset predicts poor outcome with patients subsequently requiring biologic therapy irrespective of clinical classification, and finally that integration of histological and molecular signatures into a clinical prediction model enhances sensitivity/specificity for predicting whether patients will require biologic therapy.

The data show a lower percentage of patients requiring biologic therapy in RA2010+/RA1987− group, in line with the ACR/EULAR 2010 criteria enabling an earlier diagnosis and thus efficacious treatment. However, it is also possible that this group has a milder pathology from the beginning.

Although synovial pathotypes per se do not appear to distinguish between patients at risk of developing PD rather than SL disease. However when applying 12 month biologic requirement as a prognostic outcome we demonstrated that patients with a lympho-myeloid pathotype with a dense synovial infiltrate enriched in B cells and significant upregulation of T/B cell genes at disease onset predicted requirement for subsequent biologic therapy and critically that this was independent of disease duration. The current study demonstrates that, at 12-months follow-up, a significantly higher proportion of patients classified as lympho-myeloid pathotype required biologic therapy. The study also calls into question the current dogma surrounding “an early window of opportunity” for all patients with RA, suggesting that pathotype rather than simply disease duration influences outcome and that intensive therapeutic regimens should be targeted to poor prognostic pathotypes. This notion is supported by the demonstration that the integration of synovial histological and molecular markers into a clinical prediction model for biologics use improves sensitivity/specificity from from 78.8% to 89-90% independently from disease duration.

Discrepancy with previously reported data suggesting that synovial heterogeneity does not relate to clinical phenotypes, maybe explained by the fact that in our study the majority of biopsies were performed on small joints while in that cohort arthroscopic biopsy was restricted to patients with mainly large joint involvement and, thus, a potential selection bias. Additionally, the paired histological and molecular data in the largest biopsy-driven early arthritis cohort reported to date ensured internal validation and high classification accuracy.

Our results are robust and suggest that the introduction of the new RA2010 classification criteria brings additional clinical and biological heterogeneity into early patient classification compared to the 1987 criteria with limited ability of RA2010 criteria alone to predict poor outcome. The demonstration that the integration of synovial pathobiological markers into clinical algorithms predicting poor outcome (requirement for biologic therapy) independent of disease duration suggests that the “window of opportunity” is wider than 6 months and early stratification of biologic therapies according to poor prognostic synovial pathobiological subtypes at disease onset may improve the outcome of these patients.

All publications mentioned in the above specification are herein incorporated by reference. Various modifications and variations of the disclosed methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for identifying a subject requiring treatment with a biologic therapy for rheumatoid arthritis, the method comprising the steps:
(a) determining the level of one or more biomarkers in one or more samples obtained from the subject, wherein the one or more biomarkers are selected from Table 1; and
(b) comparing the level of the one or more biomarkers to one or more corresponding reference values;
wherein the levels of the one or more biomarkers compared to the corresponding reference values are indicative of the requirement for treatment with a biologic therapy for rheumatoid arthritis.

2. The method of claim 1, wherein the one or more biomarkers comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or all 72 biomarkers from Table 1.

3. The method of claim 1 or 2, wherein the one or more biomarkers are selected from Table 2 and the levels of the one or more biomarkers are increased compared to the corresponding reference values, optionally wherein the one or more biomarkers comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or all 49 biomarkers from Table 2.

4. The method of any preceding claim, wherein the one or more biomarkers are selected from Table 3 and the levels of the one or more biomarkers are decreased compared to the corresponding reference values, optionally wherein the one or more biomarkers comprise at least 2, 3, 4, 5, 6, 7, 8, 9, 10, 11, 12, 13, 14, 15, 16, 17, 18, 19, 20, 21, 22 or all 23 biomarkers from Table 3.

5. The method of any preceding claim, wherein the step of determining the levels of the one or more biomarkers comprises determining the levels of gene expression of the one or more biomarkers.

6. The method of any preceding claim, wherein the level is a nucleic acid level, optionally wherein the nucleic acid level is an mRNA level.

7. The method of claim any preceding claim, wherein the level of the one or more biomarkers is determined by direct digital counting of nucleic acids, RNA-seq, RT-qPCR, qPCR, multiplex qPCR or RT-qPCR, microarray analysis, or a combination thereof.

8. The method of any preceding claim, wherein the subject has not been previously treated with a Disease-Modifying Anti-Rheumatic Drug (DMARD) or a biologic therapy for rheumatoid arthritis.

9. The method of any preceding claim, wherein the subject has presented one or more symptoms of rheumatoid arthritis for less than 1 year.

10. The method of any preceding claim, wherein the sample is a synovial sample, optionally wherein the sample is a synovial tissue sample or a synovial fluid sample.

11. The method of any preceding claim, wherein the method further comprises administering to the subject a biologic therapy for rheumatoid arthritis when the subject is identified as requiring treatment with a biologic therapy for rheumatoid arthritis.

12. The method of any preceding claim, wherein the biologic therapy is a B cell antagonist, a Janus kinase (JAK) antagonist, a tumour necrosis factor (TNF) antagonist, a decoy TNF receptor, a T cell costimulatory signal antagonist, an IL-1 receptor antagonist, an IL-6 receptor antagonist, or a combination thereof.

13. The method of any preceding claim, wherein the biologic therapy is an anti-TNF-alpha therapy or an anti-CD20 therapy.

14. The method of any preceding claim, wherein the biologic therapy is selected from the group consisting of adalimumab, infliximab, certolizumab pegol, golimumab, rituximab, ocrelizumab, veltuzumab, ofatumumab, tocilizumab and tofacitinib, or a combination thereof.

15. The method of any preceding claim, wherein the method further comprises the step of determining whether the subject exhibits a lympho-myeloid pathotype.

16. A method of treating rheumatoid arthritis, the method comprising administering to the subject an effective amount of a biologic therapy for rheumatoid arthritis, wherein the subject has been identified as having a requirement for treatment with a biologic therapy for rheumatoid arthritis by a method of any preceding claim.
